(19) United States
(12) Patent Application Publication
(10) Pub. No.: US 2024/0131038 A1
(43) Pub. Date: Apr. 25, 2024

(54) ANTI-INFLAMMATORY COMPOSITIONS COMPRISING CANNABIDIOL, DELTA-9-TETRAHYDROCANNABINOL AND LINALOOL

(71) Applicant: Cymra Life Sciences Limited, Sydney (AU)

(72) Inventors: Joel Ernest George Hardy, Curl Curl (AU); Simon Kenneth Pettinger, Chittaway Point (AU); John Montgomery, Burraneer (AU); Matthew Turner, Emu Heights (AU)

(73) Assignee: Cymra Life Sciences Limited, Sydney (AU)

(21) Appl. No.: 18/265,239

(22) PCT Filed: Dec. 6, 2021
(86) PCT No.: PCT/AU2021/051454

§ 371 Date: Jun. 2, 2023

(30) Foreign Application Priority Data
Dec. 4, 2020 (AU) 2020904491

(51) Int. Cl. A61K 31/00; A61K 31/045; A61P 29/00

(52) U.S. Cl. CPC A61K 31/658; A61K 31/045; A61P 29/00

(57) ABSTRACT

The present disclosure relates generally to pharmaceutical compositions comprising cannabidiol (CBD), Δ-9-tetrahydrocannabinol (THC) and linalool. In some embodiments, the pharmaceutical compositions are useful in the treatment of an inflammatory condition, e.g., useful for the treatment of pain.

### FIELD

The present disclosure relates generally to pharmaceutical compositions comprising cannabidiol (CBD), Δ-9-tetrahydrocannabinol (THC) and linalool. In some embodiments, the pharmaceutical compositions are useful in the treatment of an inflammatory condition, e.g., useful for the treatment of pain.

### RELATED APPLICATION

This application claims priority from Australian Provisional Patent Application No. 2020904491 filed on 4 Dec. 2020, the entire content of which is hereby incorporated by reference.

### BACKGROUND

Cannabis has been widely used throughout history for its medicinal properties. More recently, however, cannabis has been regarded as an illicit recreational drug. Selective breeding for this purpose has led to the development of Cannabis sativa cultivars that produce high levels of Δ⁹-tetrahydrocannabinol (THC), being the main psychoactive component of cannabis. As the medicinal properties of cannabis are becoming clearer, other cannabinoids and different classes of molecules produced by Cannabis sativa are emerging as compounds that may elicit significant therapeutic effects.

Whilst the effects of individual compounds in cannabis are important for the development of isolated drug products and to scientifically justify the effects of complex extracts, this is removed from the traditional use of cannabis (e.g., consumption of whole plant extracts). There is growing anecdotal evidence that the interactions between the chemical components of cannabis enhances their biological effect synergistically, in what has been termed “the entourage effect”. However, there is currently limited and contradictory scientific evidence to support the entourage effect in cannabis. For example, it has been demonstrated that the use of a botanical drug preparation results in an improvement in the potency of anti-tumor effects as compared to that use of pure THC (Blasco-Benito et al., 2018, Biochemical Pharmacology, 157: 285-293). By contrast, it has also been shown that terpenes commonly found in Cannabis sativa do not potentiate cannabinoid receptor signaling (via the CB₁ and CB₂ receptors) when combined with THC (Santiago et al., 2019, Cannabis and Cannabinoid Research, 4(3): 165-176).

Given the diversity and therapeutic potential of cannabis, it has been suggested that cannabis, or cannabis-derived compounds, may be used for the treatment of a broad range of conditions, such as multiple sclerosis, cancer, pain and epilepsy. In particular, there is significant therapeutic potential for the use of cannabis-derived compounds for the treatment of chronic conditions that currently have limited or no pharmacological treatment options.

Chronic inflammation is a contributing factor to many prevalent ageing-related diseases, such as acute and chronic neurodegenerative diseases, degenerative musculoskeletal diseases, cardiovascular diseases, diabetes, cancer and pain. Pain is a sensory experience that can be elicited by many different stimuli and includes neuropathic, nociceptive, and the more recently defined, nociplastic pain. Pain is a major symptom in many medical conditions that can significantly interfere with quality of life and general function.

Diagnosis of pain is based on subjective characterization, according to duration, intensity, type (e.g., dull, burning or stabbing), source or location in the body. Acute pain generally resolves without treatment or responds to simple measures such as rest or administration of an analgesic. However, when pain persists and becomes intractable, it is generally characterized as chronic pain, in which pain is no longer considered a symptom, but an illness in itself.

The management of nociceptive, neuropathic and nociplastic pain has been difficult to achieve, largely due to the complex pathophysiochemical and clinical manifestations of the different pain types.

Although there are numerous therapies available for nociceptor-induced pain, such as treatment with opioid and non-steroidal anti-inflammatory drugs (NSAIDs), these therapies are often unsatisfactory, including because treatment is required over extended periods due to the emergence of tolerance and adverse side effects. For example, common side effects associated with opioids include constipation, nausea, sedation, respiratory depression, myoclonus, urinary retention, confusion, hallucinations and dizziness. In addition, extended administration typically leads to drug tolerance, resulting in the need for administration of increased levels of drugs, thereby further exacerbating the side effects of the drugs.

Unlike nociceptive pain, neuropathic and nociplastic pain do not respond to usual analgesics, such as paracetamol and NSAIDs. Further, the identification of effective therapies for the treatment of neuropathic and nociplastic pain has proven difficult due to the distinct pathophysiochemical mechanisms of with neuropathic and nociplastic pain relative to nociceptive pain. For example, nociplastic pain is generally considered to involve alterations of nociceptive processing, most likely within the central nervous system (CNS), such as enhanced central excitability and/or diminished central inhibition, i.e., central sensitization. Nociplastic pain can occur independently of peripheral nociceptor active, however, some conditions involve both nociceptive and nociplastic pain mechanism (e.g., peripheral and central sensitization) to varying degrees along a continuum, such as non-specific back/neck pain, low back pain, knee osteoarthritis, fibromyalgia and temporomandibular disorder.

By contrast, neuropathic pain is typically caused by a lesion or disease of the somatosensory nervous system. The somatosensory nervous system allows for the perception of touch, pressure, pain, temperature, position, movement and vibration. Lesions or diseases of the somatosensory nervous system can lead to altered and disordered transmission of sensory signals into the spinal cord and brain, which can occur in common conditions associated with neuropathic pain, including post-herpetic neuralgia, trigeminal neuralgia, painful radiculopathy, diabetic neuropathy, HIV infection, leprosy, amputation, peripheral nerve injury pain and stroke. Due to the nature of this pathophysiology, the clinical management of neuropathic pain has traditionally focused on the treatment of symptoms, as the cause of the pain can rarely be treated.

Therefore, there remains an urgent need for improved pharmacological approaches for the treatment of inflammatory conditions, including pain (e.g., chronic pain).

### SUMMARY

In an aspect of the present disclosure, there is provided a pharmaceutical composition comprising a cannabinoid and a terpene, wherein the cannabinoid is enriched for cannabidiol (CBD) and Δ-9-tetrahydrocannabinol (THC), or pharmaceutically acceptable salts thereof, and wherein the terpene is enriched for linalool, or a pharmaceutically acceptable salt thereof.

In another aspect of the present disclosure, there is provided a pharmaceutical composition comprising a cannabinoid and a terpene, wherein the cannabinoid is enriched for CBD and THC, or pharmaceutically acceptable salts thereof, and wherein the terpene is enriched for linalool, or a pharmaceutically acceptable salt thereof for use in the treatment of an inflammatory condition.

In another aspect of the present disclosure, there is provided a method for the treatment or prevention of an inflammatory condition, the method comprising administering to a subject in need thereof an effective amount of a pharmaceutical composition comprising a cannabinoid and a terpene, wherein the cannabinoid is enriched for CBD and THC, or pharmaceutically acceptable salts thereof, and wherein the terpene is enriched for linalool, or a pharmaceutically acceptable salt thereof.

In another aspect of the present disclosure, there is provided use of a cannabinoid and a terpene in the manufacture of medicament for the treatment or prevention of an inflammatory condition, wherein the cannabinoid is enriched for CBD and THC, or pharmaceutically acceptable salts thereof, and wherein the terpene is enriched for linalool, or a pharmaceutically acceptable salt thereof.

### BRIEF DESCRIPTION OF THE DRAWINGS

Embodiments of the disclosure are described herein, by way of non-limiting example only, with reference to the accompanying drawings.

FIG. 1 shows the dose response of CBD, THC and linalool (μg/mL; x-axis) on nitric oxide (NO) release (%; y-axis) in lipopolysaccharide (LPS)-stimulated RAW264.7 macrophages.

FIG. 2 shows the dose response of combinations of CBD (C), THC (T) and linalool (L) (μg/mL; x-axis) on NO release (%; y-axis) in LPS-stimulated RAW264.7 macrophages.

FIG. 3 shows the dose response of a combination of CBD (C), THC (T) and linalool (L) at a ratio of 25:10:10 (μg/mL; x-axis) in comparison to the dose response of CBD alone on NO release (%; y-axis) in LPS-stimulated RAW264.7 macrophages.

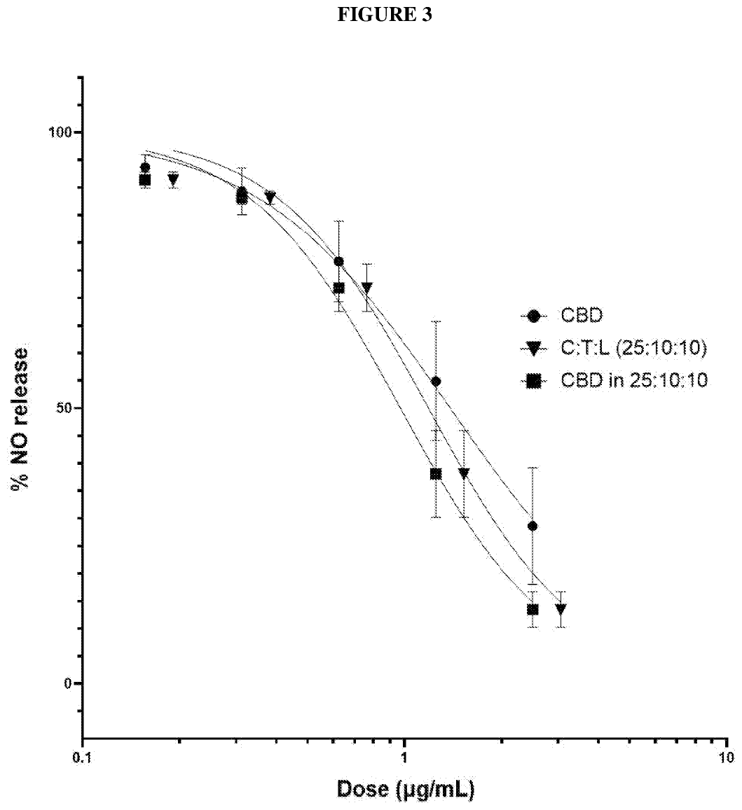

FIG. 4 shows the dose response of CBD, THC and linalool (μg/mL; x-axis) on TNF-α release (%; y-axis) in LPS-stimulated RAW264.7 macrophages.

FIG. 5 shows the dose response of combinations of CBD (C), THC (T) and linalool (L) (μg/mL; x-axis) on TNF-α release (%; y-axis) in LPS-stimulated RAW264.7 macrophages.

FIG. 6 shows the dose response of CBD, THC and linalool (μg/mL; x-axis) on IL-6 release (%; y-axis) in LPS-stimulated RAW264.7 macrophages.

FIG. 7 shows the dose response of combinations of CBD (C), THC (T) and linalool (L) (μg/mL; x-axis) on IL-6 release (%; y-axis) in LPS-stimulated RAW264.7 macrophages.

FIG. 8 shows the dose response of a combination of CBD (C), THC (T) and linalool (L) at a ratio of 25:10:10 (μg/mL; x-axis) in comparison to the dose response of CBD alone on IL-6 release (%; y-axis) in LPS-stimulated RAW264.7 macrophages.

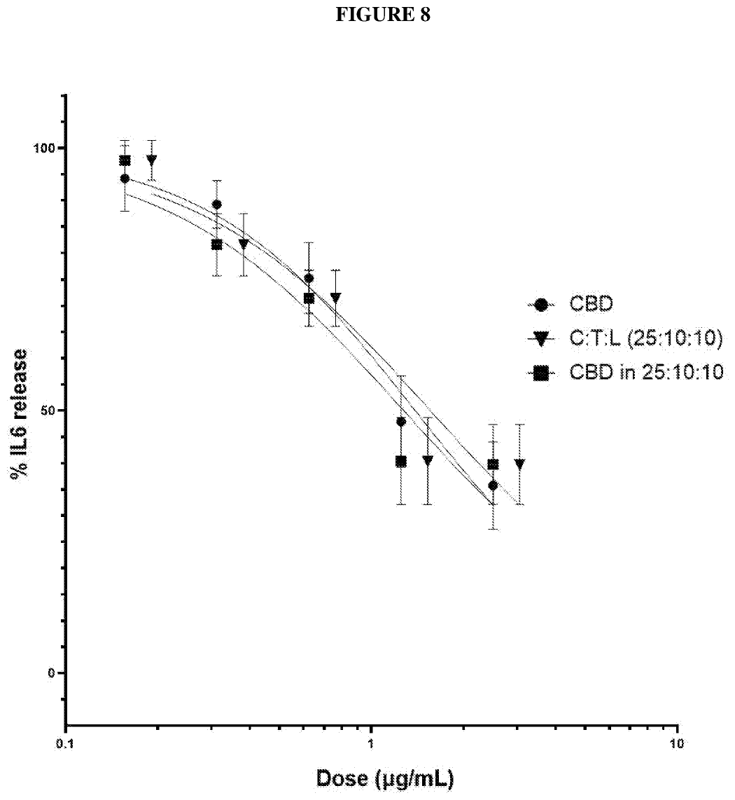

### DETAILED DESCRIPTION

Unless defined otherwise, all technical and scientific terms used herein have the same meaning as commonly understood by those of ordinary skill in the art to which the disclosure belongs. Any materials and method similar or equivalent to those described herein can be used to practice the present invention.

Throughout this specification, unless the context requires otherwise, the word “comprise”, or variations such as “comprises” or “comprising”, will be understood to imply the inclusion of the stated element or integer or group of elements or integers but not the exclusion of any other element or integer or group of elements or integers.

The phrase “consisting of” means including, and limited to, whatever follows the phrase “consisting of”. Thus, the phrase “consisting of” indicates that the listed elements are required or mandatory, and that no other elements may be present. The phrase “consisting essentially of” means including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein the singular forms “a”, “an” and “the” include plural aspects unless the context clearly dictates otherwise. Thus, for example, reference to “a compound” includes a single compound, as well as two or more compounds; reference to “an agent” includes one agent, as well as two or more agents; and so forth.

The term “about” will be understood by persons skilled in the art and will vary to some extent depending on the context in which it is used. If there are uses of the term that are not clear to persons skilled in the art, given the context which it is used, “about” will mean up to plus or minus 10% of the particular term.

The present disclosure is predicated, at least in part, on the inventor's surprising finding that pharmaceutical compositions comprising cannabidiol (CBD), Δ-9-tetrahydrocannabinol (THC) and linalool (i.e., CBD+THC+linalool) synergize to provide anti-inflammatory activity, useful for the treatment or prevention of inflammatory conditions, e.g., sepsis, traumatic injury, ischemia, asthma, burns, irritable bowel syndrome, Alzheimer's disease, cancer, major depression, arthritis, multiple sclerosis and pain. Each of the CBD, THC and linalool combinations disclosed herein exhibited synergistic inhibition of NO, TNF-α and IL-6 across a range of different weight-to-weight (w/w) ratios. For example, the combination of CBD, THC and linalool has been shown to synergize to the extent that it makes it possible to reduce the dose of CBD, while still eliciting a therapeutic effect. In certain embodiments, the combination of CBD, THC and linalool is useful to reduce pain and ameliorate common symptoms/co-morbidities of pain (e.g., sleep quality, anxiety). This combination advantageously mitigates the adverse effects otherwise associated with the administration of THC (i.e., intoxication). These findings have been reduced to practice in the preparation of pharmaceutical compositions comprising CBD, THC and linalool.

Accordingly, in an aspect disclosed herein, there is provided a pharmaceutical composition comprising CBD or a pharmaceutically acceptable salt thereof, THC or a pharmaceutically acceptable salt thereof, and linalool or a pharmaceutically acceptable salt thereof. In another aspect, there is provided a pharmaceutical composition comprising a cannabinoid and a terpene, wherein the cannabinoid is enriched for CBD and THC, or pharmaceutically acceptable salts thereof, and wherein the terpene is enriched for linalool, or a pharmaceutically acceptable salt thereof.

### Cannabidiol (CBD)

“Cannabidiol” or “CBD” is a cannabinoid produced by plants of the genus Cannabis. CBD has antagonist activity on agonists of the CB1 and CB2 receptors and acts as an inverse agonist of the CB1 and CB2 receptors.

CBD is synthesized in cannabis plants as cannabidiolic acid (CBDA), which decarboxylates to CBD (Table 1). While some decarboxylation may occur in the plant, decarboxylation typically occurs post-harvest and is increased by exposing plant material to heat (Sanchez and Verpoote, 2008, Plant Cell Physiology, 49(12): 1767-82). Decarboxylation is usually achieved by drying and/or heating the plant material. Persons skilled in the art would be familiar with methods by which decarboxylation of CBDA can be promoted, illustrative examples of which include air-drying, combustion, vaporization, curing, heating and baking. The decarboxylated CBD will typically bind to and/or stimulate, directly or indirectly, cannabinoid receptors including CB1 and/or CB2.

CBD may be extracted from any suitable plant parts including leaves, flowers or stems and may be produced by any suitable means known to those skilled in the art. For example, CBD extracts may be produced by extraction with supercritical or subcritical CO₂, or by volatilization of plant material with a heated gas. Illustrative examples of methods used to extract CBD and other cannabinoids from plant material include the methods described in U.S. patent Ser. No. 10/189,762 and WO 2004/016277.

In an embodiment, the CBD is synthetic CBD.

Synthesized CBD is particularly useful for pharmaceutical development as it is largely free from contaminants. A number of methods for the synthesis of CBD are known in the art, illustrative examples of which include methods for the synthesis of CBD as described in U.S. Pat. No. 10,059,682.

CBD is a chiral compound, although only the (−) CBD enantiomer is present in cannabis plants.

The term “enantiomer” as used herein refers to asymmetric molecules that can exist in two different isomeric forms, which have different configurations in space. An enantiomer can rotate plane-polarized light and is, therefore, optically active. Two different enantiomers of the same compound will rotate plane-polarized light in the opposite direction, thus the light can be rotated to the left or counterclockwise for a hypothetical observer (i.e., “levorotatory” or “−”) or it can be rotated to the right or clockwise (i.e., “dextrorotatory” or “+”).

In an embodiment, the synthesized CBD is a racemic mixture, comprising the (−) CBD enantiomer and the (+) CBD enantiomer.

In an embodiment, the synthetic CBD consists of the (−) CBD enantiomer.

The present disclosure further contemplates the use of pharmaceutically acceptable salts of CBD. Suitable pharmaceutically acceptable salts of CBD would be known to persons skilled in the art, illustrative examples of which include salts or esters prepared from pharmaceutically acceptable non-toxic bases or acids, including inorganic bases or acids and organic bases or acids, which would be known to persons skilled in the art.

The present disclosure further contemplates the use of functional derivatives of CBD. Suitable functional derivatives of CBD would be known to persons skilled in the art, illustrative examples of which include 7-OH-CBD (7-hydrocannabidiol), methoxylated CBD derivatives (e.g., CBDM, or 2-methoxycannabidiol and CBDD, or 2,6-dimethoxycannabidiol), cannabidiorcol (CBD-C₁) and the CBD derivatives described by Morales et al. (2017, Frontiers in Pharmacology, 8: 422).

As used herein, the term “enriched for” means that the CBD and THC, or pharmaceutically acceptable salts thereof, and linalool, or pharmaceutically acceptable salt thereof, are substantial components of the cannabinoid and terpene fractions, respectively, of the pharmaceutical compositions described herein. In an embodiment, a cannabinoid enriched for CBD and THC, or pharmaceutically acceptable salts thereof, comprise at least 50%, preferably at least 60%, preferably at least 70%, preferably at least 80%, preferably at least 90%, preferably at least 95%, or preferably at least 99% CBD and THC, or pharmaceutically acceptable salts thereof, by weight of total cannabinoids. It is to be understood that the remainder of the cannabinoid fraction or component of the pharmaceutical compositions described herein may comprise other cannabinoids, including non-CBD and non-THC cannabinoids.

In an embodiment, the pharmaceutical composition comprises from about 1 mg/mL to about 100 mg/mL CBD, or a pharmaceutically acceptable salt thereof, preferably from about 10 mg/mL to about 75 mg/mL CBD, or a pharmaceutically acceptable salt thereof (e.g., 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, 25 mg/mL, 26 mg/mL, 27 mg/mL, 28 mg/mL, 29 mg/mL, 30 mg/mL, 31 mg/mL, 32 mg/mL, 33 mg/mL, 34 mg/mL, 35 mg/mL, 36 mg/mL, 37 mg/mL, 38 mg/mL, 39 mg/mL, 40 mg/mL, 41 mg/mL, 42 mg/mL, 43 mg/mL, 44 mg/mL, 45 mg/mL, 46 mg/mL, 47 mg/mL, 48 mg/mL, 49 mg/mL, 50 mg/mL, 51 mg/mL, 52 mg/mL, 53 mg/mL, 54 mg/mL, 55 mg/mL, 56 mg/mL, 57 mg/mL, 58 mg/mL, 59 mg/mL, 60 mg/mL, 61 mg/mL, 62 mg/mL, 63 mg/mL, 64 mg/mL, 65 mg/mL, 66 mg/mL, 67 mg/mL, 68 mg/mL, 69 mg/mL, 70 mg/mL, 71 mg/mL, 72 mg/mL, 73 mg/mL, 74 mg/mL, 75 mg/mL, 76 mg/mL, 77 mg/mL, 78 mg/mL, 79 mg/mL, 80 mg/mL, 81 mg/mL, 82 mg/mL, 83 mg/mL, 84 mg/mL, 85 mg/mL, 86 mg/mL, 87 mg/mL, 88 mg/mL, 89 mg/mL, 90 mg/mL, 91 mg/mL, 92 mg/mL, 93 mg/mL, 94 mg/mL, 95 mg/mL, 96 mg/mL, 97 mg/mL, 98 mg/mL, 99 mg/mL, or 100 mg/mL CBD, or a pharmaceutically acceptable salt thereof).

Thus, in an embodiment the pharmaceutical composition comprises from about 1 mg/mL to about 100 mg/mL CBD, or a pharmaceutically acceptable salt thereof, preferably about 1 mg/mL, preferably about 2 mg/mL, preferably about 3 mg/mL, preferably about 4 mg/mL, preferably about 5 mg/mL, preferably about 6 mg/mL, preferably about 7 mg/mL, preferably about 8 mg/mL, preferably about 9 mg/mL, preferably about 10 mg/mL, preferably about 11 mg/mL, preferably about 12 mg/mL, preferably about 13 mg/mL, preferably about 14 mg/mL, preferably about 15 mg/mL, preferably about 16 mg/mL, preferably about 17 mg/mL, preferably about 18 mg/mL, preferably about 19 mg/mL, preferably about 20 mg/mL, preferably about 21 mg/mL, preferably about 22 mg/mL, preferably about 23 mg/mL, preferably about 24 mg/mL, preferably about 25 mg/mL, preferably about 26 mg/mL, preferably about 27 mg/mL, preferably about 28 mg/mL, preferably about 29 mg/mL, preferably about 30 mg/mL, preferably about 31 mg/mL, preferably about 32 mg/mL, preferably about 33 mg/mL, preferably about 34 mg/mL, preferably about 35 mg/mL, preferably about 36 mg/mL, preferably about 37 mg/mL, preferably about 38 mg/mL, preferably about 39 mg/mL, preferably about 40 mg/mL, preferably about 41 mg/mL, preferably about 42 mg/mL, preferably about 43 mg/mL, preferably about 44 mg/mL, preferably about 45 mg/mL, preferably about 46 mg/mL, preferably about 47 mg/mL, preferably about 48 mg/mL, preferably about 49 mg/mL, preferably about 50 mg/mL, preferably about 51 mg/mL, preferably about 52 mg/mL, preferably about 53 mg/mL, preferably about 54 mg/mL, preferably about 55 mg/mL, preferably about 56 mg/mL, preferably about 57 mg/mL, preferably about 58 mg/mL, preferably about 59 mg/mL, preferably about 60 mg/mL, preferably about 61 mg/mL, preferably about 62 mg/mL, preferably about 63 mg/mL, preferably about 64 mg/mL, preferably about 65 mg/mL, preferably about 66 mg/mL, preferably about 67 mg/mL, preferably about 68 mg/mL, preferably about 69 mg/mL, preferably about 70 mg/mL, preferably about 71 mg/mL, preferably about 72 mg/mL, preferably about 73 mg/mL, preferably about 74 mg/mL, preferably about 75 mg/mL, preferably about 76 mg/mL, preferably about 77 mg/mL, preferably about 78 mg/mL, preferably about 79 mg/mL, preferably about 80 mg/mL, preferably about 81 mg/mL, preferably about 82 mg/mL, preferably about 83 mg/mL, preferably about 84 mg/mL, preferably about 85 mg/mL, preferably about 86 mg/mL, preferably about 87 mg/mL, preferably about 88 mg/mL, preferably about 89 mg/mL, preferably about 90 mg/mL, preferably about 91 mg/mL, preferably about 92 mg/mL, preferably about 93 mg/mL, preferably about 94 mg/mL, preferably about 95 mg/mL, preferably about 96 mg/mL, preferably about 97 mg/mL, preferably about 98 mg/mL, preferably about 99 mg/mL, or more preferably about 100 mg/mL CBD, or a pharmaceutically acceptable salt thereof.

In a preferred embodiment, the pharmaceutical composition comprises about 25 mg/mL CBD, or a pharmaceutically acceptable salt thereof.

### Δ-9-Tetrahydrocannabinol (THC)

“Δ-9-tetrahydrocannabinolic acid” or “THCA” is synthesized in cannabis plants from the cannabigerolic acid (CBGA) precursor by THCA synthase (Table 2). THCA decarboxylates to the neutral form “Δ-9-tetrahydrocannabinol” or “THC”, which is associated with psychoactive effects of cannabis as primarily mediated by its activation of CB1G-protein coupled receptors, which result in a decrease in the concentration of cyclic AMP (cAMP) through the inhibition of adenylate cyclase. THC also exhibits partial agonist activity at the cannabinoid receptors CB1 and CB2. CB1 is mainly associated with the central nervous system, while CB2 is expressed predominantly in the cells of the immune system. As a result, THC is also associated with relaxation, fatigue, appetite stimulation, and alteration of the visual, auditory and olfactory senses. Furthermore, more recent studies have indicated that THC mediates an anti-cholinesterase action, which may suggest its use for the treatment of Alzheimer's disease and myasthenia (Eubanks et al., 2006, Molecular Pharmaceuticals, 3(6): 773-7).

THC may be extracted from any suitable plant parts including leaves, flowers or stems and may be produced by any suitable means known to those skilled in the art. For example, THC extracts may be produced by extraction with supercritical or subcritical CO₂, or by volatilization of plant material with a heated gas. Illustrative examples of methods used to extract THC and other cannabinoids from plant material include the methods described in U.S. patent Ser. No. 10/189,762 and WO 2004/016277.

In an embodiment, the THC described herein is synthetic THC.

Synthesized THC is particularly useful for pharmaceutical development as it can be prepared largely free from contaminants. A number of methods for the synthesis of THC are known in the art, illustrative examples of which include methods for the synthesis of THC (e.g., dronabinol) as described in U.S. Pat. Nos. 7,323,576 and 5,227,537, and U.S. patent application Ser. No. 11/840,585.

The present disclosure further contemplates the use of pharmaceutically acceptable salts of THC. Suitable pharmaceutically acceptable salts of THC would be known to persons skilled in the art, illustrative examples of which include salts or esters prepared from pharmaceutically acceptable non-toxic bases or acids, including inorganic bases or acids and organic bases or acids, which would be known to persons skilled in the art.

The present disclosure further contemplates the use of functional derivatives of THC. Suitable functional derivatives of THC would be known to persons skilled in the art, illustrative examples of which include 11-hydroxy-THC.

In an embodiment, the pharmaceutical composition comprises from about 1 mg/mL to about 100 mg/mL THC, or a pharmaceutically acceptable salt thereof, preferably from about 5 mg/mL to about 30 mg/mL THC (e.g., 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, 25 mg/mL, 26 mg/mL, 27 mg/mL, 28 mg/mL, 29 mg/mL, 30 mg/mL, 31 mg/mL, 32 mg/mL, 33 mg/mL, 34 mg/mL, 35 mg/mL, 36 mg/mL, 37 mg/mL, 38 mg/mL, 39 mg/mL, 40 mg/mL, 41 mg/mL, 42 mg/mL, 43 mg/mL, 44 mg/mL, 45 mg/mL, 46 mg/mL, 47 mg/mL, 48 mg/mL, 49 mg/mL, 50 mg/mL, 51 mg/mL, 52 mg/mL, 53 mg/mL, 54 mg/mL, 55 mg/mL, 56 mg/mL, 57 mg/mL, 58 mg/mL, 59 mg/mL, 60 mg/mL, 61 mg/mL, 62 mg/mL, 63 mg/mL, 64 mg/mL, 65 mg/mL, 66 mg/mL, 67 mg/mL, 68 mg/mL, 69 mg/mL, 70 mg/mL, 71 mg/mL, 72 mg/mL, 73 mg/mL, 74 mg/mL, 75 mg/mL, 76 mg/mL, 77 mg/mL, 78 mg/mL, 79 mg/mL, 80 mg/mL, 81 mg/mL, 82 mg/mL, 83 mg/mL, 84 mg/mL, 85 mg/mL, 86 mg/mL, 87 mg/mL, 88 mg/mL, 89 mg/mL, 90 mg/mL, 91 mg/mL, 92 mg/mL, 93 mg/mL, 94 mg/mL, 95 mg/mL, 96 mg/mL, 97 mg/mL, 98 mg/mL, 99 mg/mL, or 100 mg/mL THC, or a pharmaceutically acceptable salt thereof).

Thus, in an embodiment the pharmaceutical composition comprises from about 1 mg/mL to about 100 mg/mL THC, or a pharmaceutically acceptable salt thereof, preferably about 1 mg/mL, preferably about 2 mg/mL, preferably about 3 mg/mL, preferably about 4 mg/mL, preferably about 5 mg/mL, preferably about 6 mg/mL, preferably about 7 mg/mL, preferably about 8 mg/mL, preferably about 9 mg/mL, preferably about 10 mg/mL, preferably about 11 mg/mL, preferably about 12 mg/mL, preferably about 13 mg/mL, preferably about 14 mg/mL, preferably about 15 mg/mL, preferably about 16 mg/mL, preferably about 17 mg/mL, preferably about 18 mg/mL, preferably about 19 mg/mL, preferably about 20 mg/mL, preferably about 21 mg/mL, preferably about 22 mg/mL, preferably about 23 mg/mL, preferably about 24 mg/mL, preferably about 25 mg/mL, preferably about 26 mg/mL, preferably about 27 mg/mL, preferably about 28 mg/mL, preferably about 29 mg/mL, preferably about 30 mg/mL, preferably about 31 mg/mL, preferably about 32 mg/mL, preferably about 33 mg/mL, preferably about 34 mg/mL, preferably about 35 mg/mL, preferably about 36 mg/mL, preferably about 37 mg/mL, preferably about 38 mg/mL, preferably about 39 mg/mL, preferably about 40 mg/mL, preferably about 41 mg/mL, preferably about 42 mg/mL, preferably about 43 mg/mL, preferably about 44 mg/mL, preferably about 45 mg/mL, preferably about 46 mg/mL, preferably about 47 mg/mL, preferably about 48 mg/mL, preferably about 49 mg/mL, preferably about 50 mg/mL, preferably about 51 mg/mL, preferably about 52 mg/mL, preferably about 53 mg/mL, preferably about 54 mg/mL, preferably about 55 mg/mL, preferably about 56 mg/mL, preferably about 57 mg/mL, preferably about 58 mg/mL, preferably about 59 mg/mL, preferably about 60 mg/mL, preferably about 61 mg/mL, preferably about 62 mg/mL, preferably about 63 mg/mL, preferably about 64 mg/mL, preferably about 65 mg/mL, preferably about 66 mg/mL, preferably about 67 mg/mL, preferably about 68 mg/mL, preferably about 69 mg/mL, preferably about 70 mg/mL, preferably about 71 mg/mL, preferably about 72 mg/mL, preferably about 73 mg/mL, preferably about 74 mg/mL, preferably about 75 mg/mL, preferably about 76 mg/mL, preferably about 77 mg/mL, preferably about 78 mg/mL, preferably about 79 mg/mL, preferably about 80 mg/mL, preferably about 81 mg/mL, preferably about 82 mg/mL, preferably about 83 mg/mL, preferably about 84 mg/mL, preferably about 85 mg/mL, preferably about 86 mg/mL, preferably about 87 mg/mL, preferably about 88 mg/mL, preferably about 89 mg/mL, preferably about 90 mg/mL, preferably about 91 mg/mL, preferably about 92 mg/mL, preferably about 93 mg/mL, preferably about 94 mg/mL, preferably about 95 mg/mL, preferably about 96 mg/mL, preferably about 97 mg/mL, preferably about 98 mg/mL, preferably about 99 mg/mL, or more preferably about 100 mg/mL THC, or a pharmaceutically acceptable salt thereof.

In a preferred embodiment, the pharmaceutical composition comprises about 10 mg/mL THC, or a pharmaceutically acceptable salt thereof.

### Linalool

“Linalool” is a monoterpene alcohol commonly found as a major volatile component of the essential oils of several aromatic plant species, e.g., Cannabis sativa, lavender and coriander. Linalool occurs naturally as two stereoisomers: “(R)-(−)-linalool” or “licareol” and “(S)-(+)-linalool” or “coriandrol”, which exhibit different sensorial and bioactive properties. The term “linalool” as used herein encompasses one or both of the stereoisomers of linalool.

Linalool may be extracted from suitable plant parts including leaves, flowers, wood or fruits and may be produced by any suitable means known to those skilled in the art. For example, Soxhlet extraction, hydrodistillation and accelerated solvent extraction (ACE). Illustrative examples of methods used to extract linalool from plant material include the methods described by, e.g., Oliver, 2003, Journal of Essential Oil Research, 15(1): 31-33 and Zhang et al., 2016, Scientific Discovery, 4(2): 75.

As noted elsewhere herein, the term “enriched for” means that the CBD and THC, or pharmaceutically acceptable salts thereof, and linalool, or a pharmaceutically acceptable salt thereof, are substantial components of the cannabinoid and terpene fractions, respectively, of the pharmaceutical compositions described herein. For example, a terpene enriched for linalool, wherein the terpene is enriched for linalool, or a pharmaceutically acceptable salt thereof, means that the terpene fraction or component of the pharmaceutical composition comprises at least 50% linalool, preferably at least 60% linalool, preferably at least 70% linalool, preferably at least 80% linalool, preferably at least 90% linalool, preferably at least 95% linalool, or preferably at least 99% linalool by weight of total terpenes. It is to be understood that the remainder of the terpene fraction or component of the compositions described herein may comprise other terpenes.

In an embodiment, the pharmaceutical composition comprises from about 0.1 mg/mL to about 100 mg/mL linalool, or a pharmaceutically acceptable salt thereof (e.g., 0.1 mg/mL, 0.15 mg/mL, 0.2 mg/mL, 0.25 mg/mL, 0.3 mg/mL, 0.35 mg/mL, 0.4 mg/mL, 0.45 mg/mL, 0.5 mg/mL, 0.55 mg/mL, 0.6 mg/mL, 0.65 mg/mL, 0.7 mg/mL, 0.75 mg/mL, 0.8 mg/mL, 0.85 mg/mL, 0.9 mg/mL, 0.95 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, 25 mg/mL, 26 mg/mL, 27 mg/mL, 28 mg/mL, 29 mg/mL, 30 mg/mL, 31 mg/mL, 32 mg/mL, 33 mg/mL, 34 mg/mL, 35 mg/mL, 36 mg/mL, 37 mg/mL, 38 mg/mL, 39 mg/mL, 40 mg/mL, 41 mg/mL, 42 mg/mL, 43 mg/mL, 44 mg/mL, 45 mg/mL, 46 mg/mL, 47 mg/mL, 48 mg/mL, 49 mg/mL, 50 mg/mL, 51 mg/mL, 52 mg/mL, 53 mg/mL, 54 mg/mL, 55 mg/mL, 56 mg/mL, 57 mg/mL, 58 mg/mL, 59 mg/mL, 60 mg/mL, 61 mg/mL, 62 mg/mL, 63 mg/mL, 64 mg/mL, 65 mg/mL, 66 mg/mL, 67 mg/mL, 68 mg/mL, 69 mg/mL, 70 mg/mL, 71 mg/mL, 72 mg/mL, 73 mg/mL, 74 mg/mL, 75 mg/mL, 76 mg/mL, 77 mg/mL, 78 mg/mL, 79 mg/mL, 80 mg/mL, 81 mg/mL, 82 mg/mL, 83 mg/mL, 84 mg/mL, 85 mg/mL, 86 mg/mL, 87 mg/mL, 88 mg/mL, 89 mg/mL, 90 mg/mL, 91 mg/mL, 92 mg/mL, 93 mg/mL, 94 mg/mL, 95 mg/mL, 96 mg/mL, 97 mg/mL, 98 mg/mL, 99 mg/mL, or 100 mg/mL linalool, or a pharmaceutically acceptable salt thereof).

In an embodiment, the pharmaceutical composition comprises from about 0.1 mg/mL to about 100 mg/mL linalool or a pharmaceutically acceptable salt thereof, preferably about 0.1 mg/mL, preferably about 0.15 mg/mL, preferably about 0.2 mg/mL, preferably about 0.25 mg/mL, preferably about 0.3 mg/mL, preferably about 0.35 mg/mL, preferably about 0.4 mg/mL, preferably about 0.45 mg/mL, preferably about 0.5 mg/mL, preferably about 0.55 mg/mL, preferably about 0.6 mg/mL, preferably about 0.65 mg/mL, preferably about 0.7 mg/mL, preferably about 0.75 mg/mL, preferably about 0.8 mg/mL, preferably about 0.85 mg/mL, preferably about 0.9 mg/mL, preferably about 0.95 mg/mL, preferably about 1 mg/mL, preferably about 2 mg/mL, preferably about 3 mg/mL, preferably about 4 mg/mL, preferably about 5 mg/mL, preferably about 6 mg/mL, preferably about 7 mg/mL, preferably about 8 mg/mL, preferably about 9 mg/mL, preferably about 10 mg/mL, preferably about 11 mg/mL, preferably about 12 mg/mL, preferably about 13 mg/mL, preferably about 14 mg/mL, preferably about 15 mg/mL, preferably about 16 mg/mL, preferably about 17 mg/mL, preferably about 18 mg/mL, preferably about 19 mg/mL, preferably about 20 mg/mL, preferably about 21 mg/mL, preferably about 22 mg/mL, preferably about 23 mg/mL, preferably about 24 mg/mL, preferably about 25 mg/mL, preferably about 26 mg/mL, preferably about 27 mg/mL, preferably about 28 mg/mL, preferably about 29 mg/mL, preferably about 30 mg/mL, preferably about 31 mg/mL, preferably about 32 mg/mL, preferably about 33 mg/mL, preferably about 34 mg/mL, preferably about 35 mg/mL, preferably about 36 mg/mL, preferably about 37 mg/mL, preferably about 38 mg/mL, preferably about 39 mg/mL, preferably about 40 mg/mL, preferably about 41 mg/mL, preferably about 42 mg/mL, preferably about 43 mg/mL, preferably about 44 mg/mL, preferably about 45 mg/mL, preferably about 46 mg/mL, preferably about 47 mg/mL, preferably about 48 mg/mL, preferably about 49 mg/mL, preferably about 50 mg/mL, preferably about 51 mg/mL, preferably about 52 mg/mL, preferably about 53 mg/mL, preferably about 54 mg/mL, preferably about 55 mg/mL, preferably about 56 mg/mL, preferably about 57 mg/mL, preferably about 58 mg/mL, preferably about 59 mg/mL, preferably about 60 mg/mL, preferably about 61 mg/mL, preferably about 62 mg/mL, preferably about 63 mg/mL, preferably about 64 mg/mL, preferably about 65 mg/mL, preferably about 66 mg/mL, preferably about 67 mg/mL, preferably about 68 mg/mL, preferably about 69 mg/mL, preferably about 70 mg/mL, preferably about 71 mg/mL, preferably about 72 mg/mL, preferably about 73 mg/mL, preferably about 74 mg/mL, preferably about 75 mg/mL, preferably about 76 mg/mL, preferably about 77 mg/mL, preferably about 78 mg/mL, preferably about 79 mg/mL, preferably about 80 mg/mL, preferably about 81 mg/mL, preferably about 82 mg/mL, preferably about 83 mg/mL, preferably about 84 mg/mL, preferably about 85 mg/mL, preferably about 86 mg/mL, preferably about 87 mg/mL, preferably about 88 mg/mL, preferably about 89 mg/mL, preferably about 90 mg/mL, preferably about 91 mg/mL, preferably about 92 mg/mL, preferably about 93 mg/mL, preferably about 94 mg/mL, preferably about 95 mg/mL, preferably about 96 mg/mL, preferably about 97 mg/mL, preferably about 98 mg/mL, preferably about 99 mg/mL, or more preferably about 100 mg/mL linalool or a pharmaceutically acceptable salt thereof.

In a preferred embodiment, the pharmaceutical composition comprises 1 mg/mL linalool, or a pharmaceutically acceptable salt thereof.

In a preferred embodiment, the pharmaceutical composition comprises about 10 mg/mL THC, or a pharmaceutically acceptable salt thereof, about 25 mg/mL CBD, or a pharmaceutically acceptable salt thereof, and about 1 mg/mL linalool, or a pharmaceutically acceptable salt thereof.

The pharmaceutical compositions of the present disclosure suitably comprise combinations of cannabinoids (i.e., CBD and THC) and a terpene (i.e., linalool) that synergize to inhibit the production, release or activity of inflammatory molecules (e.g., NO, TNF-α, and IL-6).

The terms “level”, “content”, “concentration” and the like, are used interchangeably herein to describe an amount of the referenced compound, and may be represented in absolute terms (e.g., mg/g, mg/mL, etc.) or in relative terms, such as a ratio.

In accordance with certain embodiments, pharmaceutical compositions comprising CBD, THC and linalool, or pharmaceutically acceptable salts of any of the foregoing, may be defined by reference to the ratio of each active compound in the composition. In an embodiment, the composition may also be described as comprising THC, CBD and linalool at a ratio of about 10:25:1 (THC:CBD:linalool) or CBD, THC and linalool at a ratio of about 25:10:1 (CBD:THC:linalool). A ratio of cannabinoid and terpene is intended to encompass a reasonable level of variance, e.g., a ratio of “about” may encompass a level of variation being plus or minus 10%. In certain embodiments, the ratio of cannabinoid and terpene may be presented by reference to decimals of each component, e.g., 25:10:7.5, 25:10:7.6, 25:10:7.7, 25:10:7.8, 25:10:7.9 (CBD:THC:linalool), and so on and so forth.

In an embodiment, the pharmaceutical composition comprises a CBD:THC:linalool ratio of from about 25:10:0.5 to about 25:10:20 (w/w), preferably about 25:10:0.5, preferably about 25:10:1, preferably about 25:10:1.5, preferably about 25:10:2, preferably about 25:10:2.5, preferably about 25:10:3, preferably about 25:10:3.5, preferably about 25:10:4, preferably about 25:10:4.5, preferably about 25:10:5, preferably about 25:10:5.5, preferably about 25:10:6, preferably about 25:10:6.5, preferably about 25:10:7, preferably about 25:10:7.5, preferably about 25:10:8, preferably about 25:10:8.5, preferably about 25:10:9, preferably about 25:10:9.5, preferably about 25:10:10, preferably about 25:10:10.5, preferably about 25:10:11, preferably about 25:10:11.5, preferably about 25:10:12, preferably about 25:10:12.5, preferably about 25:10:13, preferably about 25:10:13.5, preferably about 25:10:14, preferably about 25:10:14.5, preferably about 25:10:15, preferably about 25:10:15.5, preferably about 25:10:16, preferably about 25:10:16.5, preferably about 25:10:17, preferably about 25:10:17.5 preferably about 25:10:18, preferably about 25:10:18.5, preferably about 25:10:19, preferably about 25:10:19.5 or more preferably about 25:10:20 (w/w).

In an embodiment, the pharmaceutical composition comprises a CBD:THC:linalool ratio of about 25:10:10 (w/w).

In another embodiment, the pharmaceutical composition comprises a CBD:THC:linalool ratio of about 25:10:1 (w/w).

In an embodiment, the pharmaceutical composition further comprises one or more pharmaceutically acceptable carriers, diluents and excipients.

Suitable pharmaceutically acceptable carriers, diluents or excipients would be known to persons skilled in the art, illustrative examples of which include inert diluents (e.g., calcium carbonate, lactose, calcium phosphate or sodium phosphate), granulating and disintegrating agents (e.g., corn starch or alginic acid), binding agents (e.g., starch, gelatin or acacia), lubricating agents (e.g., magnesium stearate, stearic acid or talc) and material to delay disintegration and absorption in the gastrointestinal tract and thereby provide a sustained action over a longer period (e.g., glyceryl monostearate or glyceryl distearate). Coating may also be performed using techniques described in the U.S. Pat. Nos. 4,256,108, 4,160,452, and 4,265,874 to form osmotic therapeutic tablets for control release.

The pharmaceutical composition may be administered in dosage unit and in formulations containing conventional non-toxic pharmaceutically acceptable carriers, adjuvants, and vehicles. Formulations include transdermal, aerosol, nasal spray, sublingual spray, liposomal, nanoparticle, microparticle, polymer-based, dispersion, suspension, powder, microspheres, carrier-mediated, and encapsulation.

Pharmaceutical compositions disclosed herein may be prepared according to conventional methods well known in the pharmaceutical and nutraceutical industries, such as those described in Remington's Pharmaceutical Handbook (Mack Publishing Co., NY, USA) using suitable excipients, diluents and fillers.

In an embodiment, the pharmaceutical composition is formulated for oral administration.

Pharmaceutical compositions suitable for oral administration would be known to persons skilled in the art, illustrative examples of which include liquid, oil, tablets and capsules. The term “oral administration” as used herein broadly encompasses formulations for sublingual and buccal administration.

In an embodiment, the pharmaceutical composition is in oil form.

Pharmaceutical compositions for oral administration may contain one or more additional agents selected from the group of sweetening agents, flavoring agents, coloring agents and preserving agents in order to produce pharmaceutically elegant and palatable preparations. Suitable sweeteners include sucrose, lactose, glucose, aspartame or saccharin. Suitable disintegrating agents include corn starch, methylcellulose, polyvinylpyrrolidone, xanthan gum, bentonite, alginic acid or agar. Suitable flavoring agents include peppermint oil, oil of wintergreen, cherry, orange or raspberry flavoring. Suitable preservatives include sodium benzoate, vitamin E, alphatocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Pharmaceutical compositions suitable for oral administration may be presented as discrete units (i.e., dosage forms), each containing a predetermined amount of each component of the composition as a powder, tablet, capsule, granules, as a solution or a suspension in an aqueous liquid or non-aqueous liquid, or as an emulsion.

As described elsewhere herein, the pharmaceutical compositions may be formulated for administration as separate unit dosage forms for administration. Suitable dosage forms for oral administration would be known to persons skilled in the art, illustrative examples of which include tablets, aqueous or oily suspensions, lozenges, troches, powders, granules, emulsions, capsules, liquids, syrups or elixirs.

Oral administration has been demonstrated to be an effective administration route for CBD and THC (reviewed by Millar et al., 2018, Frontiers in Pharmacology, 9: 1365 and Poyatos et al., 2020, Medicina (Kaunas), 56(6): 309). Similarly, oral administration of linalool has also been demonstrated to be effective for absorption with high bioavailability (Nöldner et al., 2013, Planta Medica, 79(13)).

In an embodiment, the pharmaceutical composition as disclosed herein is suitable for the treatment of an inflammatory condition.

### Inflammatory Conditions

As used herein, the term “inflammatory condition” typically refers to a condition characterized by inflammation, or the complex biological response to a noxious stimulus such as damage, auto-immunity, or an infection by a microbial pathogen and/or virus. The clinical features of an inflammatory condition are likely to depend on the noxious stimulus (or stimuli), but is typically characterized by any one or more of heat, pain, redness and swelling of the affected organ or tissue. The inflammatory condition may be acute or chronic.

In an embodiment, the inflammatory condition is selected from the group consisting of sepsis, traumatic injury, ischemia, asthma, burns, irritable bowel syndrome, Alzheimer's disease, cancer, major depression, arthritis, multiple sclerosis and pain.

In an embodiment, the inflammatory condition is associated with an increase or upregulation in the level of an inflammatory cytokine selected from the group consisting of NO, IL-6, TNF-α, and combinations of the foregoing.

Inflammatory conditions associated with an increase or upregulation in the level of NO, IL-6, and/or TNF-α would be known to persons skilled in the art, illustrative examples of which include irritable bowel disease (as described by, e.g., Neurath, 2014, Nature Reviews Immunology, 14: 329-342; Papadakis and Targan, 2000, The Annual Review of Medicine, 51: 289-298), pain (as described by, e.g., Zhang, 2007, International Anesthesiology Clinics, 45: 27-37), and asthma (as described by, e.g., Rincon and Irvin, 2012, International Journal of Biological Sciences, 8: 1281-1290; Thomas, 2001, Immunology & Cell Biology, 79: 132-140).

In an embodiment, the inflammatory condition is pain.

### Pain

The term “pain” as used herein refers to an unpleasant sensory and emotional experience associated with, or resembling that associated with actual or potential tissue damage. There are three clinically recognized types of pain—nociceptive pain, neuropathic pain and nociplastic pain (Merskey and Bogduk, 1994, Classification of Chronic Pain, Second Edition, IASP Task Force on Taxonomy).

“Nociceptive pain” is the result of stimulation of the sensory nerve fibers, as detected by nociceptors around the body that respond to mechanical or physical damage. Nociceptive pain serves a protective function by warning of tissue damage, to cause withdrawal from the noxious stimulus, such as thermal damage (e.g., burns or frostbite) or mechanical trauma (e.g., laceration or pressure).

“Neuropathic pain” is caused by a primary lesion, malfunction or dysfunction in the peripheral or central nervous system. Neuropathic pain has no protective effect and can develop days or months after an injury or after resolution of a disease state, and is frequently long lasting and chronic.

“Nociplastic pain” is caused by altered nociception with no corresponding or clear evidence or actual or threatened tissue damage causing the activation of peripheral nociceptors or evidence for disease or lesion of the somatosensory system causing the pain.

In an embodiment, the pain is nociceptive pain.

In an embodiment, the pain is neuropathic pain.

In an embodiment, the pain is nociplastic pain.

“Acute pain” usually lasts for short periods (e.g., a few hours or days), and will typically disappear upon cessation of the underlying stimulus.

“Chronic pain” lasts for longer periods (e.g., weeks or months), and can persist even in the absence of an underlying stimulus.

In an embodiment, the pharmaceutical composition is for use in the treatment of acute pain. In an embodiment, the acute pain is acute nociceptive pain. In an embodiment, the acute pain is acute neuropathic pain. In an embodiment, the acute pain is acute nociplastic pain.

In an embodiment, the pharmaceutical composition is for use in the treatment of chronic pain. In an embodiment, the chronic pain is chronic nociceptive pain. In an embodiment, the chronic pain is chronic neuropathic pain. In an embodiment, the chronic pain is chronic nociplastic pain.

In an embodiment, the pharmaceutical composition is for use in the treatment of chronic back pain. In an embodiment, the pharmaceutical composition is for use in the treatment of chronic neck pain. In an embodiment, the pharmaceutical composition is for use in the treatment of chronic back and neck pain.

The present disclosure also contemplates the use of the compositions described herein for the treatment of pain, wherein the pain is not associated with an inflammatory condition. Such compositions may alternatively be referred to herein as analgesic compositions. The types of pain not associated with an inflammatory condition will be known to persons skilled in the art, illustrative examples of which include mechanical pain and non-inflammatory musculoskeletal pain.

In an embodiment, the pharmaceutical composition is for use in the treatment of pain.

### Methods of Treatment

In an aspect disclosed herein, there is provided a method for the treatment or prevention of an inflammatory condition, the method comprising administering a therapeutically effective amount of the pharmaceutical composition as disclosed herein to a subject in need thereof.

The terms “treat”, “treating”, “treatment”, and the like refer to any and all methods that remedy, prevent, hinder, retard, ameliorate, reduce, delay or reverse or otherwise inhibit the severity of the condition (e.g., an inflammatory condition) or of one or more symptoms thereof in a subject. Treatment does not necessarily imply that a patient is treated until total recovery. Inflammatory conditions are characterized by multiple symptoms/comorbidities (e.g., sleep quality, anxiety, quality of life), and thus the treatment need not necessarily remedy, prevent, hinder, retard, ameliorate, reduce, delay or reverse all of said symptoms/comorbidities. Methods of the present disclosure may involve “treating” an inflammatory condition in terms of reducing, preventing or ameliorating the occurrence of a highly undesirable event or symptom associated with an inflammatory condition or an outcome of the progression of the inflammatory condition, but may not of itself prevent the initial occurrence of the event, symptom, comorbidity or outcome. Accordingly, treatment includes amelioration of the symptoms of an inflammatory condition or preventing or otherwise reducing the risk of developing symptoms/comorbidities of an inflammatory condition.

The term “subject” as used herein refers to any mammal, including livestock and other farm animals (such as cattle, goats, sheep, horses, pigs and chickens), performance animals (such as racehorses), companion animals (such as cats and dogs), laboratory test animals and humans. In an embodiment, the subject is a human.

Pharmaceutical compositions comprising CBD, THC and linalool, including pharmaceutically acceptable salts of any of the foregoing, will suitably be administered to the subject in need thereof in a therapeutically effective amount. As used herein, the term “therapeutically effective amount” or “effective amount” typically refers to an amount of CBD, THC and linalool that is sufficient to affect one or more beneficial or desired therapeutic outcomes (e.g., reduction or amelioration of the symptoms of an inflammatory condition, e.g., pain). Said beneficial or desired therapeutic outcomes may be subjectively measured using clinical instruments known in the art, illustrative examples of which include the detection of altered levels of immunological biomarkers and the Numerical Rating Scale (NRS) of Pain Intensity (2000, Spine, 25: 3200-3202), Brief Pain Inventory-Short Form (Cleeland, 1991). The reduction or amelioration of the symptoms/comorbidities of an inflammatory condition may also be assessed using secondary indicators of beneficial or desired therapeutic outcomes (i.e., secondary indicia), e.g., improvements to physical functioning, sleep quality, emotional function, and quality of life. Said secondary indicators of beneficial or desired therapeutic outcomes may be subjectively measured using clinical instruments known in the art, illustrative examples of which include the Depression Anxiety Stress Scale (Lovibond and Lovibond, 1995, Manual for the Depression Anxiety Stress Scales, 2^{nd} edition, Sydney: Psychology Foundation), Medical Outcomes of Sleep Survey (Shahid et al., in Shahid et al. (eds), 2012 STOP, THAT and One Hundred Other Sleep Scales, Springer, New York, pp 219-222), Pittsburgh Sleep Quality Index (Buysse et al., 1989, Psychiatry Research, 28(2): 193-213), Roland Morris Disability Questionnaire (Roland and Morris, 1983, Spine, 8: 141-144), Hospital Anxiety and Depression Scale (Zigmond and Snaith, 1983, Acta Psychiatrica Scandinavia, 67(6): 361-70), EuroQol 5D (EuroQol Research Foundation, 2019, EQ-5D-5L User Guide), Short Form Medical Outcomes 12 (Ware et al., 1996, Med Care, 34: 220-233), and Patient Global Impression of Change (Hurst and Bolton, 2004, Journal of Manipulative & Physiological Therapeutics, 27: 26-35).

Changes in the symptoms or severity of an inflammatory condition, e.g., pain, including as measured by any of the clinical instruments described elsewhere herein, may be expressed using any appropriate statistical measure to demonstrate the magnitude of the reduction in the symptoms or severity of the inflammatory condition. In an embodiment, the methods disclosed herein reduce in the symptoms or severity of the inflammatory condition by at least 10%, preferably at least 20%, preferably at least 30%, preferably at least 40%, preferably at least 50%, preferably at least 60%, preferably at least 70%, preferably at least 80%, preferably at least 90%, or more preferably at least 100% as compared to a subject with an inflammatory condition who has not been administered a composition comprising CBD, THC and linalool.

In an embodiment, the symptoms or severity of the inflammatory condition is reduced by at least 30% as compared to a subject with the inflammatory condition who has not been administered a pharmaceutical composition comprising CBD, THC and linalool.

In an embodiment, the symptoms or severity of the inflammatory condition is reduced by at least 50% as compared to a subject with the inflammatory condition who has not been administered a pharmaceutical composition comprising CBD, THC and linalool.

An effective amount can be provided in one or more administrations. The exact amount required may vary depending on factors such as the nature and severity of the inflammatory condition to be treated, the age and general health of the subject.

In an embodiment, the effective amount comprises from about 1 mg to about 100 mg of CBD, preferably from about 12.5 mg to about 75 mg of CBD (e.g., 1 mg, 2 mg, 3 mg, 4 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 34 mg, 34.5 mg, 35 mg, 35.5 mg, 36 mg, 36.5 mg, 37 mg, 37.5 mg, 38 mg, 38.5 mg, 39 mg, 39.5 mg, 40 mg, 40.5 mg, 41 mg, 41.5 mg, 42 mg, 42.5 mg, 43 mg, 43.5 mg, 44 mg, 44.5 mg, 45 mg, 45.5 mg, 46 mg, 46.5 mg, 47 mg, 47.5 mg, 48 mg, 48.5 mg, 49 mg, 49.5 mg, 50 mg, 50.5 mg, 51 mg, 51.5 mg, 52 mg, 52.5 mg, 53 mg, 53.5 mg, 54 mg, 54.5 mg, 55 mg, 55.5 mg, 56 mg, 56.5 mg, 57 mg, 57.5 mg, 58 mg, 58.5 mg, 59 mg, 59.5 mg, 60 mg, 60.5 mg, 61 mg, 61.5 mg, 62 mg, 62.5 mg, 63 mg, 63.5 mg, 64 mg, 64.5 mg, 65 mg, 65.5 mg, 66 mg, 66.5 mg, 67 mg, 67.5 mg, 68 mg, 68.5 mg, 69 mg, 69.5 mg, 70 mg, 70.5 mg, 71 mg, 71.5 mg, 72 mg, 72.5 mg, 73 mg, 73.5 mg, 74 mg, 74.5 mg, 75 mg, 75.5 mg, 76 mg, 76.5 mg, 77 mg, 77.5 mg, 78 mg, 78.5 mg, 79 mg, 79.5 mg, 80 mg, 80.5 mg, 81 mg, 81.5 mg, 82 mg, 82.5 mg, 83 mg, 83.5 mg, 84 mg, 84.5 mg, 85 mg, 85.5 mg, 86 mg, 86.5 mg, 87 mg, 87.5 mg, 88 mg, 88.5 mg, 89 mg, 89.5 mg, 90 mg, 90.5 mg, 91 mg, 91.5 mg, 92 mg, 92.5 mg, 93 mg, 93.5 mg, 94 mg, 94.5 mg, 95 mg, 95.5 mg, 96 mg, 96.5 mg, 97 mg, 97.5 mg, 98 mg, 98.5 mg, 99 mg, 99.5 mg, or 100 mg of CBD)

Thus, in an embodiment the effective amount comprises from about 1 mg to about 100 mg of CBD, preferably about 1 mg, preferably about 2 mg, preferably about 3 mg, preferably about 4 mg, preferably about 5 mg, preferably about 5.5 mg, preferably about 6 mg, preferably about 6.5 mg, preferably about 7 mg, preferably about 7.5 mg, preferably about 8 mg, preferably about 8.5 mg, preferably about 9 mg, preferably about 9.5 mg, preferably about 10 mg, preferably about 10.5 mg, preferably about 11 mg, preferably about 11.5 mg, preferably about 12 mg, preferably about 12.5 mg, preferably about 13 mg, preferably about 13.5 mg, preferably about 14 mg, preferably about 14.5 mg, preferably about 15 mg, preferably about 15.5 mg, preferably about 16 mg, preferably about 16.5 mg, preferably about 17 mg, preferably about 17.5 mg, preferably about 18 mg, preferably about 18.5 mg, preferably about 19 mg, preferably about 19.5 mg, preferably about 20 mg, preferably about 20.5 mg, preferably about 21 mg, preferably about 21.5 mg, preferably about 22 mg, preferably about 22.5 mg, preferably about 23 mg, preferably about 23.5 mg, preferably about 24 mg, preferably about 24.5 mg, preferably about 25 mg, preferably about 25.5 mg, preferably about 26 mg, preferably about 26.5 mg, preferably about 27 mg, preferably about 27.5 mg, preferably about 28 mg, preferably about 28.5 mg, preferably about 29 mg, preferably about 29.5 mg, preferably about 30 mg, preferably about 30.5 mg, preferably about 31 mg, preferably about 31.5 mg, preferably about 32 mg, preferably about 32.5 mg, preferably about 33 mg, preferably about 33.5 mg, preferably about 34 mg, preferably about 34.5 mg, preferably about 35 mg, preferably about 35.5 mg, preferably about 36 mg, preferably about 36.5 mg, preferably about 37 mg, preferably about 37.5 mg, preferably about 38 mg, preferably about 38.5 mg, preferably about 39 mg, preferably about 39.5 mg, preferably about 40 mg, preferably about 40.5 mg, preferably about 41 mg, preferably about 41.5 mg, preferably about 42 mg, preferably about 42.5 mg, preferably about 43 mg, preferably about 43.5 mg, preferably about 44 mg, preferably about 44.5 mg, preferably about 45 mg, preferably about 45.5 mg, preferably about 46 mg, preferably about 46.5 mg, preferably about 47 mg, preferably about 47.5 mg, preferably about 48 mg, preferably about 48.5 mg, preferably about 49 mg, preferably about 49.5 mg, preferably about 50 mg, preferably about 50.5 mg, preferably about 51 mg, preferably about 51.5 mg, preferably about 52 mg, preferably about 52.5 mg, preferably about 53 mg, preferably about 53.5 mg, preferably about 54 mg, preferably about 54.5 mg, preferably about 55 mg, preferably about 55.5 mg, preferably about 56 mg, preferably about 56.5 mg, preferably about 57 mg, preferably about 57.5 mg, preferably about 58 mg, preferably about 58.5 mg, preferably about 59 mg, preferably about 59.5 mg, preferably about 60 mg, preferably about 60.5 mg, preferably about 61 mg, preferably about 61.5 mg, preferably about 62 mg, preferably about 62.5 mg, preferably about 63 mg, preferably about 63.5 mg, preferably about 64 mg, preferably about 64.5 mg, preferably about 65 mg, preferably about 65.5 mg, preferably about 66 mg, preferably about 66.5 mg, preferably about 67 mg, preferably about 67.5 mg, preferably about 68 mg, preferably about 68.5 mg, preferably about 69 mg, preferably about 69.5 mg, preferably about 70 mg, preferably about 70.5 mg, preferably about 71 mg, preferably about 71.5 mg, preferably about 72 mg, preferably about 72.5 mg, preferably about 73 mg, preferably about 73.5 mg, preferably about 74 mg, preferably about 74.5 mg, preferably about 75 mg, preferably about 75.5 mg, preferably about 76 mg, preferably about 76.5 mg, preferably about 77 mg, preferably about 77.5 mg, preferably about 78 mg, preferably about 78.5 mg, preferably about 79 mg, preferably about 79.5 mg, preferably about 80 mg, preferably about 80.5 mg, preferably about 81 mg, preferably about 81.5 mg, preferably about 82 mg, preferably about 82.5 mg, preferably about 83 mg, preferably about 83.5 mg, preferably about 84 mg, preferably about 84.5 mg, preferably about 85 mg, preferably about 85.5 mg, preferably about 86 mg, preferably about 86.5 mg, preferably about 87 mg, preferably about 87.5 mg, preferably about 88 mg, preferably about 88.5 mg, preferably about 89 mg, preferably about 89.5 mg, preferably about 90 mg, preferably about 90.5 mg, preferably about 91 mg, preferably about 91.5 mg, preferably about 92 mg, preferably about 92.5 mg, preferably about 93 mg, preferably about 93.5 mg, preferably about 94 mg, preferably about 94.5 mg, preferably about 95 mg, preferably about 95.5 mg, preferably about 96 mg, preferably about 96.5 mg, preferably about 97 mg, preferably about 97.5 mg, preferably about 98 mg, preferably about 98.5 mg, preferably about 99 mg, preferably about 99.5 mg, or more preferably about 100 mg of CBD.

In an embodiment, the effective amount comprises from about 0.1 mg to about 100 mg of linalool, preferably from about 0.25 mg to about 3 mg of linalool (e.g., 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, or 100 mg of linalool).

Thus, in an embodiment the effective amount comprises from about 0.1 mg to about 100 mg of linalool, preferably about 0.1 mg, preferably about 0.15 mg, preferably about 0.2 mg, preferably about 0.25 mg, preferably about 0.3 mg, preferably about 0.35 mg, preferably about 0.4 mg, preferably about 0.45 mg, preferably about 0.5 mg, preferably about 0.55 mg, preferably about 0.6 mg, preferably about 0.65 mg, preferably about 0.7 mg, preferably about 0.75 mg, preferably about 0.8 mg, preferably about 0.85 mg, preferably about 0.9 mg, preferably about 0.95 mg, preferably about 1 mg, preferably about 2 mg, preferably about 3 mg, preferably about 4 mg, preferably about 5 mg, preferably about 6 mg, preferably about 7 mg, preferably about 8 mg, preferably about 9 mg, preferably about 10 mg, preferably about 11 mg, preferably about 12 mg, preferably about 13 mg, preferably about 14 mg, preferably about 15 mg, preferably about 16 mg, preferably about 17 mg, preferably about 18 mg, preferably about 19 mg, preferably about 20 mg, preferably about 21 mg, preferably about 22 mg, preferably about 23 mg, preferably about 24 mg, preferably about 25 mg, preferably about 26 mg, preferably about 27 mg, preferably about 28 mg, preferably about 29 mg, preferably about 30 mg, preferably about 31 mg, preferably about 32 mg, preferably about 33 mg, preferably about 34 mg, preferably about 35 mg, preferably about 36 mg, preferably about 37 mg, preferably about 38 mg, preferably about 39 mg, preferably about 40 mg, preferably about 41 mg, preferably about 42 mg, preferably about 43 mg, preferably about 44 mg, preferably about 45 mg, preferably about 46 mg, preferably about 47 mg, preferably about 48 mg, preferably about 49 mg, preferably about 50 mg, preferably about 51 mg, preferably about 52 mg, preferably about 53 mg, preferably about 54 mg, preferably about 55 mg, preferably about 56 mg, preferably about 57 mg, preferably about 58 mg, preferably about 59 mg, preferably about 60 mg, preferably about 61 mg, preferably about 62 mg, preferably about 63 mg, preferably about 64 mg, preferably about 65 mg, preferably about 66 mg, preferably about 67 mg, preferably about 68 mg, preferably about 69 mg, preferably about 70 mg, preferably about 71 mg, preferably about 72 mg, preferably about 73 mg, preferably about 74 mg, preferably about 75 mg, preferably about 76 mg, preferably about 77 mg, preferably about 78 mg, preferably about 79 mg, preferably about 80 mg, preferably about 81 mg, preferably about 82 mg, preferably about 83 mg, preferably about 84 mg, preferably about 85 mg, preferably about 86 mg, preferably about 87 mg, preferably about 88 mg, preferably about 89 mg, preferably about 90 mg, preferably about 91 mg, preferably about 92 mg, preferably about 93 mg, preferably about 94 mg, preferably about 95 mg, preferably about 96 mg, preferably about 97 mg, preferably about 98 mg, preferably about 99 mg, or more preferably about 100 mg of linalool.

In an embodiment, the effective amount comprises from about 1 mg to about 100 mg of THC, preferably from about 5 mg to about 30 mg (e.g., 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, or 100 mg of THC).

Thus, in an embodiment the effective amount comprises from about 1 mg to about 100 mg of THC, preferably about 1 mg, preferably about 2 mg, preferably about 3 mg, preferably about 4 mg, preferably about 5 mg, preferably about 6 mg, preferably about 7 mg, preferably about 8 mg, preferably about 9 mg, preferably about 10 mg, preferably about 11 mg, preferably about 12 mg, preferably about 13 mg, preferably about 14 mg, preferably about 15 mg, preferably about 16 mg, preferably about 17 mg, preferably about 18 mg, preferably about 19 mg, preferably about 20 mg, preferably about 21 mg, preferably about 22 mg, preferably about 23 mg, preferably about 24 mg, preferably about 25 mg, preferably about 26 mg, preferably about 27 mg, preferably about 28 mg, preferably about 29 mg, preferably about 30 mg, preferably about 31 mg, preferably about 32 mg, preferably about 33 mg, preferably about 34 mg, preferably about 35 mg, preferably about 36 mg, preferably about 37 mg, preferably about 38 mg, preferably about 39 mg, preferably about 40 mg, preferably about 41 mg, preferably about 42 mg, preferably about 43 mg, preferably about 44 mg, preferably about 45 mg, preferably about 46 mg, preferably about 47 mg, preferably about 48 mg, preferably about 49 mg, preferably about 50 mg, preferably about 51 mg, preferably about 52 mg, preferably about 53 mg, preferably about 54 mg, preferably about 55 mg, preferably about 56 mg, preferably about 57 mg, preferably about 58 mg, preferably about 59 mg, preferably about 60 mg, preferably about 61 mg, preferably about 62 mg, preferably about 63 mg, preferably about 64 mg, preferably about 65 mg, preferably about 66 mg, preferably about 67 mg, preferably about 68 mg, preferably about 69 mg, preferably about 70 mg, preferably about 71 mg, preferably about 72 mg, preferably about 73 mg, preferably about 74 mg, preferably about 75 mg, preferably about 76 mg, preferably about 77 mg, preferably about 78 mg, preferably about 79 mg, preferably about 80 mg, preferably about 81 mg, preferably about 82 mg, preferably about 83 mg, preferably about 84 mg, preferably about 85 mg, preferably about 86 mg, preferably about 87 mg, preferably about 88 mg, preferably about 89 mg, preferably about 90 mg, preferably about 91 mg, preferably about 92 mg, preferably about 93 mg, preferably about 94 mg, preferably about 95 mg, preferably about 96 mg, preferably about 97 mg, preferably about 98 mg, preferably about 99 mg, or more preferably about 100 mg of THC.

In accordance with the methods disclosed herein, the effective amount of CBD, THC and linalool may be administered sequentially or simultaneously, and as part of the same pharmaceutical composition or as part of separate pharmaceutical compositions (e.g., a first pharmaceutical composition comprising the effective amount CBD, a second pharmaceutical composition comprising the effective amount of THC, a third pharmaceutical composition comprising the effective amount of linalool).

By “sequential” administration it is meant there is an interval between the administration of the CBD, THC and linalool. The interval between sequential administrations may be seconds, minutes, hours or days. In a preferred embodiment, the interval between sequential administrations of CBD, THC and linalool is less than an hour, preferably less than 30 minutes, most preferably less than 1 minute. Sequential administration may be in any order.

In some embodiments, periodic re-administration of the pharmaceutical composition may be required to achieve a desirable therapeutic effect. The exact amounts and rates of administration of the pharmaceutical composition will depend on a number of factors, examples of which are described elsewhere herein, such as the subject's age, body weight, general health, sex and dietary requirements, as well as any drugs or agents used in combination or coincidental with the administration of the pharmaceutical composition. Where multiple divided doses are required, these may be administered hourly, daily, weekly, monthly or at other suitable time intervals or the dose may be proportionally reduced as indicated by the exigencies of the situation.

In an embodiment, the pharmaceutical composition is administered twice per day.

In an embodiment, the pharmaceutical composition is administered for a period of at least one week.

In an embodiment, the pharmaceutical composition is administered to the subject for a period of between 1 to 10 weeks (e.g., for 1 week, 2 weeks, 3 weeks, 4 weeks, 5 weeks, 6 weeks, 7 weeks, 8 weeks, 9 weeks, or 10 weeks).

Thus, in an embodiment, the pharmaceutical composition is administered to the subject for a period of between 1 to 10 weeks, preferably for about 1 week, preferably about 2 weeks, preferably about 3 weeks, preferably about 4 weeks, preferably about 5 weeks, preferably about 6 weeks, preferably about 7 weeks, preferably about 8 weeks, preferably about 9 weeks, or more preferably for about 10 weeks.

In an embodiment, the pharmaceutical composition is administered to the subject for a period of 5 weeks.

It is to be understood, however, that there may be instances where the pharmaceutical compositions described herein are advantageously administered to the subject over a prolonged period of time (e.g., in excess of 5 weeks), or until such time as the symptoms or severity of an inflammatory condition have been sufficiently resolved to the satisfaction of the patient and/or medical practitioner. In some embodiments, the pharmaceutical compositions described herein are administered even after the symptoms or severity of the inflammatory condition (e.g., pain) have been sufficiently resolved, including to prevent the inflammatory condition from returning. Such prophylactic uses may suitably be prescribed by medical practitioners having regard to, for example, the likelihood that the inflammatory condition will return.

In an embodiment, the method comprises administering to the subject the pharmaceutical composition as escalating daily doses comprise a first daily dose and a second daily dose, wherein the effective amount of the pharmaceutical composition administered as the second daily dose is greater than the effective amount of the pharmaceutical composition administered as the first daily dose.

The amount and frequency of administration of escalating daily doses may be determined in accordance with methods of “empiric therapy” or “empirical administration.” The person skilled in the art would appreciate that empirical administration of escalating daily doses (e.g., first daily dose, second daily dose, third daily dose, fourth daily dose, fifth daily dose, and so on) will be determined by an appropriate medically trained professional overseeing the methods of treatment described herein.

In an embodiment, the effective amount of the first daily dose comprises from about 5 mg to about 25 mg CBD, from about 0.1 mg to about 100 mg linalool and from about 1 mg to about 10 mg THC.

In an embodiment, the effective amount of the first daily dose comprises about 12.5 mg CBD, about 0.5 mg linalool and about 5 mg THC.

In an embodiment, the effective amount of the second daily dose comprising from about 20 mg to about 30 mg CBD, from about 0.5 mg to about 5 mg linalool, and from about 5 mg to about 15 mg THC.

In an embodiment, the effective amount of the second daily dose comprises about 25 mg CBD, about 1 mg linalool, and about 10 mg THC.

The time interval between the escalation from the first daily dose to the second daily dose may be daily, weekly, monthly or at other suitable time intervals or the dose may be proportionally reduced as indicated by the exigencies of the situation.

In an embodiment, the subject is administered the first daily dose for a week.

In an embodiment, the method further comprises administering to the subject a third daily dose, wherein the effective amount of the pharmaceutical composition administered as the third daily dose is greater than the effective amount of the pharmaceutical composition administered as the second daily dose.

In an embodiment, the effective amount of the third daily dose comprises from about 40 mg CBD to about 60 mg CBD, from about 1 mg linalool to about 5 mg linalool, and from about 15 mg to about 25 mg THC.

In an embodiment, the effective amount of the third daily dose comprises about 50 mg CBD, about 2 mg linalool, and about 20 mg THC.

The time interval between the escalation from the second daily dose to the third daily dose may be daily, weekly, monthly or at other suitable time intervals or the dose may be proportionally reduced as indicated by the exigencies of the situation.

In an embodiment, the subject is administered the second daily dose for a week.

In an embodiment, the method further comprises administering to the subject a fourth daily dose and/or subsequent daily dose, wherein the effective about of the pharmaceutical composition administered as the fourth daily dose is greater than the effective amount of the pharmaceutical composition administered as the third daily dose.

In an embodiment, the effective amount of the fourth daily dose comprises from about 65 mg to about 80 mg CBD, from about 1 mg to about 5 mg linalool, and from about 25 mg to about 35 mg THC. In an embodiment, the effective amount of the fourth daily dose comprises from about 65 mg to about 80 mg CBD, from about 1 mg to about 10 mg linalool, and from about 25 mg to about 35 mg THC.

In an embodiment, the effective amount of the fourth daily dose comprises about 75 mg CBD, about 3 mg linalool, and about 30 mg THC. In an embodiment, the effective amount of the fourth daily dose comprises about 75 mg CBD, about 5 mg linalool, and about 30 mg THC.

The time interval between the escalation from the third daily dose to the fourth daily dose may be daily, weekly, monthly or at other suitable time intervals or the dose may be proportionally reduced as indicated by the exigencies of the situation.

In an embodiment, the subject is administered the third daily dose for a week.

In an embodiment, the subject is administered the fourth daily dose for a week.

In an embodiment, the first daily dose, second daily dose, third daily dose and fourth daily dose may be administered to the subject as divided doses, such that, e.g., the subject is administered a first half of the daily dose in the morning and the second half of the daily dose in the evening.

It is further contemplated herein that the subject may be administered a subsequent daily dose (i.e., a fifth daily dose, a sixth daily dose, a seventh daily dose, an eighth daily dose, a ninth daily dose, a tenth daily dose, and so on and so forth), as required.

In an embodiment, the pharmaceutical composition is administered to the subject orally.

In an embodiment, the pharmaceutical composition is administered to the subject sublingually.

In an embodiment, the pharmaceutical composition is administrated to the subject sublabially (i.e., buccal administration).

In an aspect disclosed herein, there is provide a use of CBD or a pharmaceutically acceptable salt thereof, THC or a pharmaceutically acceptable salt thereof and linalool or a pharmaceutically acceptable salt thereof in the manufacture of a medicament for the treatment or prevention of an inflammatory condition.

In an embodiment, the inflammatory condition is selected from the group consisting of sepsis, traumatic injury, ischemia, asthma, burns, irritable bowel syndrome, Alzheimer's disease, cancer, major depression, arthritis, multiple sclerosis and pain.

In an embodiment, the inflammatory condition is pain. In another embodiment, the pain is chronic pain. In another embodiment the chronic pain is chronic back and/or neck pain.

In another aspect disclosed herein, there is provided a use of a cannabinoid and a terpene in the manufacture of medicament for the treatment or prevention of an inflammatory condition, wherein the cannabinoid is enriched for cannabidiol (CBD) and Δ-9-tetrahydrocannabinol (THC), or pharmaceutically acceptable salts thereof, and wherein the terpene is enriched for linalool, or a pharmaceutically acceptable salt thereof.

In an embodiment, the inflammatory condition is selected from the group consisting of sepsis, traumatic injury, ischemia, asthma, burns, irritable bowel syndrome, Alzheimer's disease, cancer, major depression, arthritis, multiple sclerosis and pain.

In an embodiment, the inflammatory condition is pain. In another embodiment, the pain is chronic pain. In another embodiment the chronic pain is chronic back and/or neck pain.

In an embodiment, the pain is chronic pain.

In an embodiment, the pain is chronic nociplastic pain.

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Unless otherwise defined, all technical and scientific terms used herein have the same meanings as commonly understood by one of ordinary skill in the art to which this invention belongs.

All patents, patent applications and publications mentioned herein are hereby incorporated by reference in their entireties.

The various embodiments enabled herein are further described by the following non-limiting examples.

### Example 1

### Anti-Inflammatory Activity

MATERIALS and methods

### Materials

Cannabinoid preparations comprising 25 mg/mL CBD, 10 mg/mL THC were prepared from pure cannabinoids obtained from a commercial supplier and solubilized in an appropriate solvent prior to in vitro analysis. Similarly, linalool preparations comprising various concentrations of linalool were prepared from pure linalool obtained from a commercial supplier and solubilized in an appropriate solvent prior to in vitro analysis.

Combinations of CBD, THC and linalool were prepared at ratios of 25:10:1 (i.e., equivalent to 25 mg/mL CBD, 10 mg/mL THC and 1 mg/mL linalool), 25:10:2 (i.e., equivalent to 25 mg/mL CBD, 10 mg/mL THC and 2 mg/mL linalool), 25:10:5 (i.e., equivalent to 25 mg/mL CBD, 10 mg/mL THC and 5 mg/mL linalool), 25:10:7.5 (i.e., equivalent to 25 mg/mL CBD, 10 mg/mL THC and 7.5 mg/mL linalool), 25:10:10 (i.e., equivalent to 25 mg/mL CBD, 10 mg/mL THC and 10 mg/mL linalool), 25:10:12.5 (i.e., equivalent to 25 mg/mL CBD, 10 mg/mL THC and 12.5 mg/mL linalool), and 25:10:15 (i.e., equivalent to 25 mg/mL CBD, 10 mg/mL THC and 15 mg/mL linalool).

### Assays

Anti-inflammatory activity was determined in lipopolysaccharide (LPS) stimulated murine macrophages, RAW264.7 cells cultured in standard cell culture media (DMEM, Fetal bovine serum 5%) and incubated in the presence or absence of different test compounds/extracts and controls. The production of inflammatory mediators, including NO, cytokines TNFα and IL-6, were measured by established methods using commercial ELISA kits (Table 3). Each sample was tested with at least 6 concentrations (using 3 replicates; i.e., n=9), with relevant internal controls (Table 3). The cytotoxicity of each sample tested was determined by MTT assay.

Assay parameters for each assay are summarized in Table 3. Briefly, for the NO, TNFα and IL-6 assays the cultured RAW264.7 cells were counted and plated (0.8×10⁵ cells/well) in 96 well plates and incubated for 48 hours. The medium was then aspirated and replaced with fresh medium followed by the addition of the test compounds. The compounds were incubated for 1 hour prior to the addition of the stimulant. The plates were then incubated for 18 hours and the supernatant analyzed for the inflammatory mediator of interest, the remaining cell viability was determined by MTT.

Positive controls were selected based on their widespread use in similar assays, including N-(3-(Aminomethyl)benzyl)acetamidine (1400W), a slow, tight binding inhibitor of inducible nitric-oxide synthase (iNOS) (Garvey et al., 1997, Journal of Biological Chemistry, 272(8): 4959-4963), and dexamethasone, a commonly used cytokine inhibitor.

### Data Analysis

The data are presented as mean and standard error of mean (SEM). The dose response curves were fit using Graph Pad Prism. The 95% confidence intervals (CI) were calculated using the whole data set and give an estimate error in the IC₅₀ value. The 95% CI for the IC₅₀ where the IC₅₀ was not reached in the testing concentrations require graphical extrapolation to calculate the IC₅₀. Where the IC₅₀ has been estimated by graphical extrapolation “˜” is used to indicate that it is an estimate and not experimentally determined, unless otherwise stated. The graphical extrapolation results in a wider 95% confidence interval.

### Synergy Calculation

The synergism combination index (CI) and isobologram IC₅₀ weightings were calculated using Compsyn software. The dose response curve generated in GraphPad prism was transformed into 15 points representing the curve. These 15 points were then entered into the Compsyn program that generated a curve to fit the data points. This method was preferable to closely replicate the complete dose response curve in the synergy program. The Compsyn fit curve was then used for synergy calculations.

A CI value of less than 0.5 is considered highly synergistic, a CI of less than 1 indicates synergy, a CI of from 1.5 to 1 is considered additive, a CI of from 4 to 1.5 is indifferent and a CI greater than 4 is considered antagonism.

### Results

### Nitric Oxide (NO) Assay

NO is a radical metabolite, which has been shown to have numerous physiological functions both as a signaling molecule and as a toxic agent in inflammation (Coleman, 2001, International Immunopharmacology, 1(8): 1397-1406). NO is derived from the oxidation of L-arginine by three types of nitric oxide synthases (NOS); the constitutive forms, neuronal NOS and endothelial NOS, and the inducible form, iNOS, originally described in murine macrophages (Nathan & Xie, 1994, Cell, 78(6): 915-918; Stuehr & Marletta, 1985, Proceedings of the National Academy of Sciences U.S.A., 82(22): 7738-7742). The inducible form is continually activated once expressed, and is therefore regulated at the transcription level by NF-κB, stimulated by inflammatory molecules like LPS and IFN-γ. The production of NO by iNOS (due to the time taken for messenger ribonucleic acid (mRNA) and protein synthesis) experiences hours of lag time before NO is produced in much higher (nM) sustained levels (Nathan & Xie, 1994, supra). The inducible form of NOS is most likely implicated in inflammation and due to the higher levels of NO produced it is more easily assessed in-vitro.

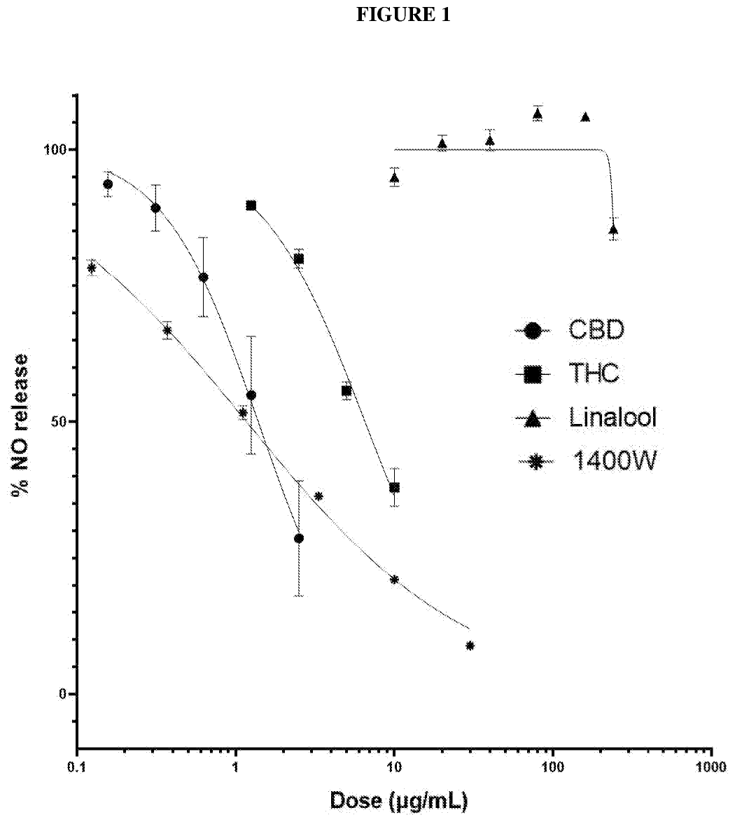

The cannabinoids (i.e., CBD, THC), linalool and mixtures (i.e., CBD+THC+linalool) were therefore assayed to determine if the blends showed synergistic inhibition on the inflammatory signaling molecule NO. The dose dependent effects of the individual cannabinoids and linalool are shown in FIG. 1. The cannabinoids and linalool tested inhibited NO release in a dose dependent manner. The most active compound for NO inhibition was CBD (Table 4). The cannabinoids were significantly more potent inhibitors of NO compared to linalool.

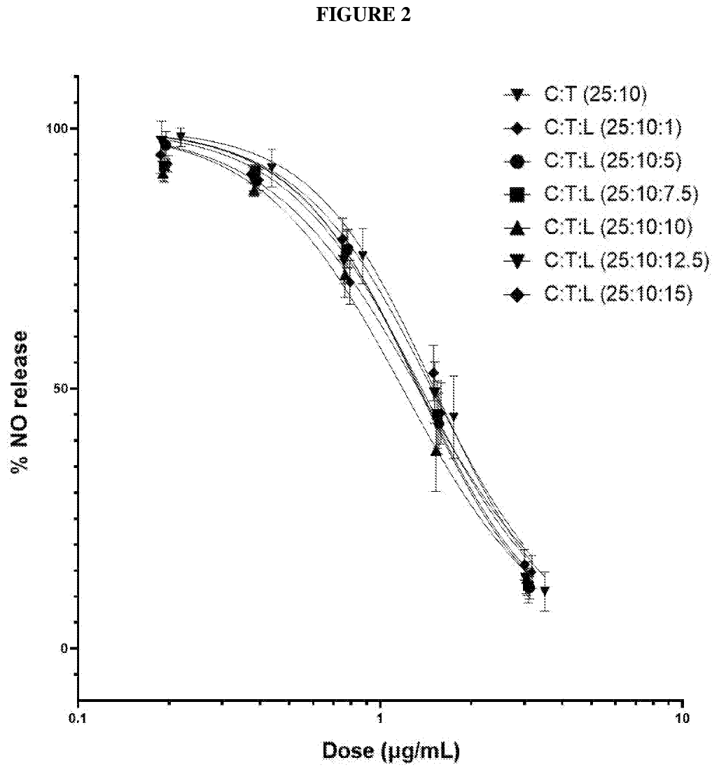

To investigate if there is a synergistic relationship between the cannabinoids and linalool they were tested in combination. The cannabinoids (i.e., CBD+THC) were combined with linalool (i.e., CBD+THC+linalool) at various ratios. The dose response results for the combinations are shown in FIG. 2.

The IC₅₀ for each pure compound and combination was calculated and is shown in Table 5. The combination index (CI) also calculated at 75% inhibition (i.e., IC₇₅) and 90% inhibition (i.e., IC₉₀). Many combinations showed synergistic interaction. The ‘best’ combinations show synergy and high potency. These combinations have been highlighted in bold (Table 6).

The synergistic effect associated with the combination of CBD, THC and linalool was observed at all ratios tested. The 25:10:10 ratio (w/w) was the most potent and synergistic combination (Table 5 and Table 6).

The combination with the lowest IC₅₀ for CBD was also the 25:10:10 ratio (w/w), where the concentration of CBD at the IC₅₀ was 0.7 μg/mL (Table 6). Accordingly, the CBD was nearly double the potency in these optimum combinations.

### Summary

All combinations comprising CBD and THC showed a synergistic anti-inflammatory effect on the release of NO from LPS stimulated RAW264.7 cells when combined with linalool at multiple ratios. The combination that resulted in the most significant CI was the 25:10:10 ratio, which was the most synergistic and potent combination.

### TNF-α Assay

TNF-α is a cell signaling protein (cytokine) involved mainly in the acute phase inflammatory response. Macrophages are the major source of TNF-α, although it can be released by many other cell types such as CD4+ lymphocytes, natural killer (NK) cells, neutrophils, mast cells, eosinophils, and neurons. TNF-α is produced by activation of MAPK and NF-κB. It acts to increase its own production and that of other inflammatory cytokines such as interleukin-1 beta (IL-β). TNF-α induces fever, apoptotic cell death, cachexia, inflammation and inhibits tumorigenesis and viral replication. TNF-α is implicated in many disease states, including, sepsis, traumatic injury, ischemia, asthma, burns, irritable bowel syndrome, Alzheimer's disease, cancer, major depression, arthritis and multiple sclerosis (see, e.g., Cairns et al., 2000, Academic Emergency Medicine, 7(8): 930-941; Dowlati et al., 2010, Biological Psychiatry, 67(5): 446-457; Swardfager et al., 2010, Biological Psychiatry, 68(10): 930-941).

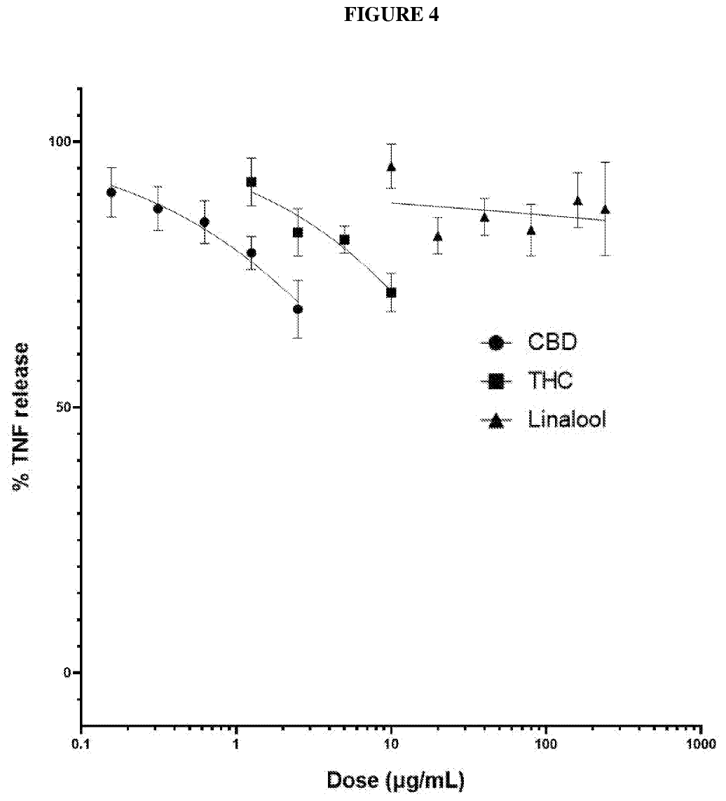

The cannabinoids (i.e., CBD, THC), linalool and mixtures (i.e., CBD+THC+linalool) were therefore assayed to determine if the blends showed synergistic inhibition on the on the inflammatory cytokine TNF-α. The dose dependent effects of the individual cannabinoids and linalool are shown in FIG. 4, which demonstrates that the cannabinoids and linalool tested inhibited TNF-α release in a dose dependent manner. The most active compound for TNF-α inhibition was CBD (Table 7). The cannabinoids were significantly more potent inhibitors of TNF-α compared to linalool.

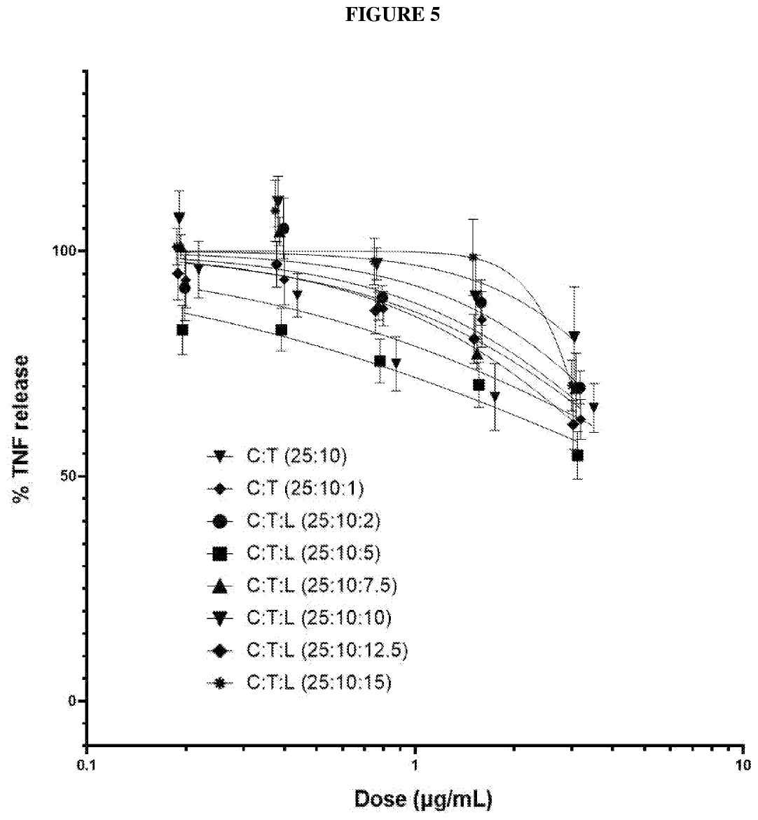

To investigate if there is a synergistic relationship between the cannabinoids and linalool they were tested in combination. The cannabinoids (i.e., CBD+THC) were combined with linalool (i.e., CBD+THC+linalool) at various ratios. The dose response results for the combinations are shown in FIG. 5.

The IC₅₀ for each pure compound and combination was calculated and is shown in Table 6. The combination index (CI) also calculated at 75% inhibition (i.e., IC₇₅) and 90% inhibition (i.e., IC₉₀). Many combinations showed synergistic interaction. The ‘best’ combinations show synergy and high potency. These combinations have been highlighted in bold (Table 8).

The synergistic effect associated with the combination of CBD, THC and linalool was observed at all ratios tested. The 25:10:12.5 ratio (w/w) was the most potent and synergistic combination (Table 8 and Table 9).

The combination with the lowest IC₅₀ for CBD was also the 25:10:12.5 ratio (w/w), where the concentration of CBD at the IC₅₀ was 0.6 μg/mL (Table 9). Accordingly, the CBD was nearly double the potency in these optimum combinations.

### Summary

All combinations comprising CBD and THC showed a synergistic anti-inflammatory effect on the release of TNF-α from LPS stimulated RAW264.7 cells when combined with linalool at multiple ratios. The combination that resulted in the most significant CI was the 25:10:12.5 ratio, which was the most synergistic and potent combination.

### IL-6 Assay

Like TNF-α, IL-6 is considered a pro-inflammatory cytokine. IL-6 is secreted by T cells and macrophages which stimulates an immune response. IL-6 is responsible for increased production of neutrophils in bone marrow. It supports the growth of B cells and is antagonistic to differentiation of T cells into regulatory T cells. It is capable of crossing the blood-brain barrier and initiating synthesis of PGE₂ in the hypothalamus, thereby changing the body's temperature set point.

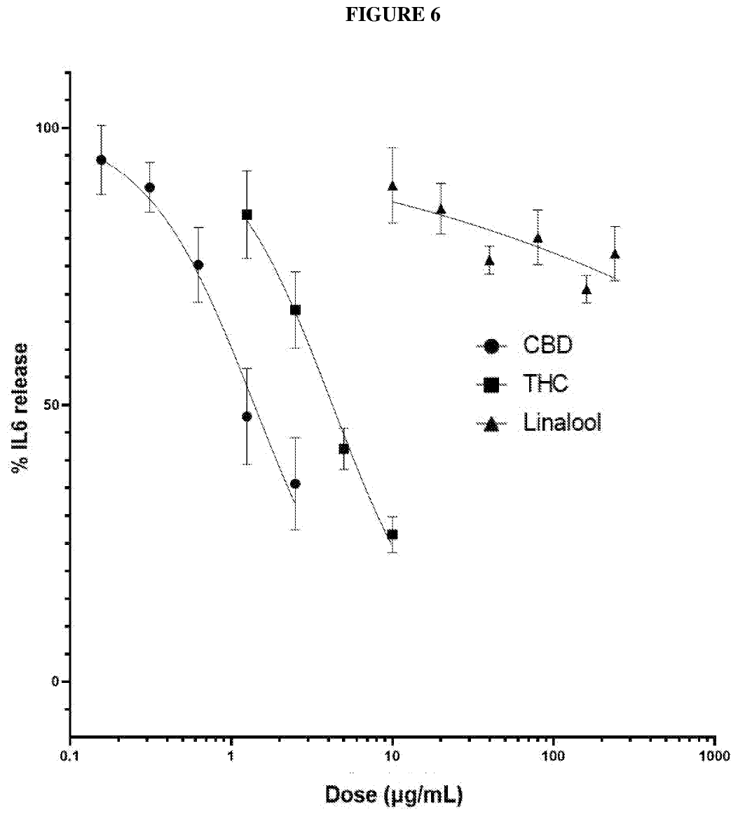

The cannabinoids (i.e., CBD, THC), linalool and mixtures (i.e., CBD+THC+linalool) were therefore assayed to determine if the blends showed synergistic inhibition on the inflammatory cytokine IL-6. The dose dependent effects of the individual cannabinoids and linalool are shown in FIG. 6, which demonstrates that the cannabinoids and linalool tested inhibited IL-6 release in a dose dependent manner. The most active compound for IL-6 inhibition was CBD (Table 10). The cannabinoids were significantly more potent inhibitors of IL-6 compared to linalool.

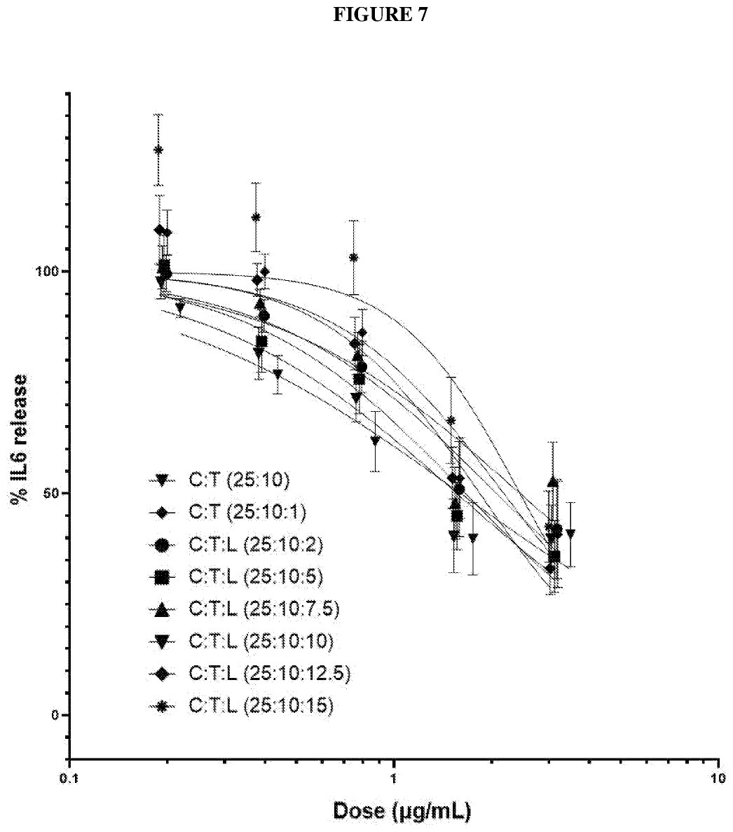

To investigate if there is a synergistic relationship between the cannabinoids and linalool they were tested in combination. The cannabinoids (i.e., CBD+THC) were combined with linalool (i.e., CBD+THC+linalool) at various ratios. The dose response results for the combinations are shown in FIG. 7.

The IC₅₀ for each pure compound and combination was calculated and is shown in Table 6. The combination index (CI) also calculated at 75% inhibition (i.e., IC₇₅) and 90% inhibition (i.e., IC₉₀). Many combinations showed synergistic interaction. The ‘best’ combinations show synergy and high potency. These combinations have been highlighted in bold (Table 11).

The synergistic effect associated with the combination of CBD, THC and linalool was observed at the 25:10:5, 25:10:10 and 25:10:12.5 ratios (w/w). The 25:10:10 ratio (w/w) was the most potent and synergistic combination (Table 11 and Table 12).

The combination with the lowest IC₅₀ for CBD was the 25:10:10 ratio (w/w), where the concentration of CBD at the IC₅₀ was 0.9 μg/mL (Table 12). Accordingly, the CBD was nearly 1.5× the potency in these optimum combinations.

### Summary

Combinations comprising CBD and THC showed a synergistic anti-inflammatory effect on the release of IL-6 from LPS stimulated RAW264.7 cells when combined with linalool at the 25:10:5, 25:10:10 and 25:10:12.5 ratios (w/w). The combination that resulted in the most significant CI was the 25:10:10 ratio, which was the most synergistic and potent combination.

### Discussion

Collectively, these data demonstrate that combinations of CBD, THC and linalool synergize to significantly inhibit inflammatory molecules in vitro. The methods described herein have been reduced to practice in methods for modulating an inflammatory response and methods for the treatment of an inflammatory condition, such as sepsis, traumatic injury, ischemia, asthma, burns, irritable bowel syndrome, Alzheimer's disease, cancer, major depression, arthritis, multiple sclerosis and pain. In this context, the combination of CBD, THC and linalool can be used to modulate inflammatory mediators, such as NO, TNF-α, and IL-6, which are known to be elevated in chronic inflammatory conditions. Further, pharmaceutical compositions comprising CBD, THC and linalool synergize to the extent that it makes it possible to reduce the dose of CBD to a level that may improve the safety profile of the pharmaceutical compositions, particularly for long-term use, while still eliciting a therapeutic effect.

### Example 2

### Clinical Studies

### Materials

Cannabis oil comprising 10 mg/mL THC, 25 mg/mL CBD and 1 mg/mL linalool for oro-buccal administration was prepared in accordance with the Therapeutic Goods (Standard for Medicinal Cannabis) (TGO 93) Order 2017 (TGO 93). The cannabis oil containing 10 mg/mL THC, 25 mg/mL CBD and 1 mg/mL linalool may be variously described herein as “10:25:1 medicinal cannabis product” and “Cybis 10:25:1.”

### Dose-Ranging Study

A Phase I/II controlled, open-label clinical trial of the buccal administration of the medicinal cannabis product comprising 10 mg/mL THC, 25 mg/mL CBD and 1 mg/mL linalool for the treatment of chronic pain will be performed to demonstrate the safety and tolerability of the 10:25:1 medicinal cannabis product at low, medium-low, medium-high and high doses. The clinical trial will also investigate the pharmacokinetics of the 10:25:1 medicinal cannabis product in single dose and repeat dose settings and to investigate any dose-response relationship in terms of pain relief and quality of life following administration of the 10:25:1 medicinal cannabis product.

Patients with moderate to severe chronic back/neck pain (i.e., chronic nociceptive, neuropathic and/or nociplastic pain), clinically characterized as having an NRS of 5-9 for more than 3 months, who have not achieved satisfactory response to non-opioid analgesics, such as paracetamol and nonsteroidal anti-inflammatory drugs (NSAIDs; e.g., ibuprofen), will be randomized into 4-arms: (i) low dose (0.5 mL), (ii) medium low dose (1.0 mL as 0.5 mL twice daily), (iii) medium high dose (2.0 mL as 1.0 mL twice daily), and (iv) high dose (3.0 mL as 1.5 mL twice daily).

The clinical trial design consists of the following:
a. On Day −7, participants cease current pain medication;
b. On Day 0, baseline assessments of pain intensity, physical function, emotional function and sleep quality will be undertaken.
c. On Day 1, participants will receive 0.5 mL the 10:25:1 medicinal cannabis product in the morning after an overnight fast. Blood samples will be collected prior to first dose then at 0.25, 0.5, 1.0, 1.5, 2.0, 2.5, 3.0, 3.5, 4.0, 5.0, 6.0, 9.0, 12.0, and 24.0 hrs after the dose for measurement of plasma concentration of CBD and THC. After the 24-hour blood sample, participants will receive 0.5 mL dose daily for a further 6 days (daily dose of 0.5 mL; Days 1-7; i.e., first daily dose). A blood sample will be taken on Day 7 before the last dose (steady state for 0.5 mL dose).
d. At the start of week 2, all participants will increase the dose to 0.5 mL twice a day (morning and evening) for 7 days (daily dose of 1.0 mL; Days 8-14; i.e., second daily dose). A blood sample will be taken on Day 14 before the last dose (steady state for 1.0 mL dose).
e. At the start of week 3, all participants will again increase the dose to 1.0 mL in the morning and 1.0 mL in the evening for 7 days (daily dose of 2.0 mL; Days 15-21; i.e., third daily dose). A blood sample will be taken on Day 21 before the last dose (steady state for 2.0 mL dose).
f. At the start of week 4, all participants will again increase the dose to 1.5 mL in the morning and 1.5 mL in the evening for 7 days (daily dose of 3.0 mL; Days 22-28; i.e., fourth daily dose). A blood sample will be taken on Day 28 before the last dose (steady state for 3.0 mL dose)
g. Patients followed up for safety assessment 7 days after last dose (Day 35)

Clinical response will be measured at baseline and on the last day of each dose level (i.e., days 0, 7, 14, 21 and 28). Rescue medication (e.g., pain medication taken prior to study, if none, immediate release tramadol 50 mg (S4)) will be available if required.

Clinical response will be measured according to the primary efficacy assessment tools of the Numerical Rating Scale (NRS) of pain intensity, as described elsewhere herein; measurement of pain interference of general activities (Brief Pain Inventory-Short Form, as described elsewhere herein); measurement of mood (Depression Anxiety Stress Scale, as described elsewhere herein); and measurement of sleep quality (Medical Outcomes of Sleep Survey, as described elsewhere herein). Additional efficacy measures of mean pain relief (change in average pain intensity from baseline), change from baseline in pain interference, change in baseline from mood, change from baseline in sleep quality, global perception of improvement and rescue medication use will also be considered.

Blood samples will also be used for analysis of plasma/serum content of CBD, linalool, and/or THC, or a metabolite of any of the foregoing, to assess bioavailability and pharmacokinetic parameters, e.g., Tₘₐₓ, Cₘₐₓ, AUC₀₋₄, AUC_{0-∞}, t½ Kₑₗ for CBD, THC, 11-OH-THC.

### Safety/Efficacy Study

A Phase II, 3-arm, double blind, placebo and active controlled, randomized clinical trial of the buccal administration of the medicinal cannabis product comprising 10 mg/mL THC, 25 mg/mL CBD and 1 mg/mL linalool for the treatment of chronic pain will be performed.

Patients with moderate to severe chronic back/neck pain (i.e., chronic nociplastic, neuropathic and/or nociceptive pain), clinically characterized as having an NRS of 5-9 for more than 3 months, who have not responded to non-opioid analgesics, such as paracetamol and NSAIDs, will be randomized into 3-arms: (i) placebo, (ii) standard pain treatment (e.g., paracetamol/codeine) and (iii) 10:25:1 medicinal cannabis product.

The clinical trial design consists of a 1 week baseline phase, a 1 week dose titration phase of the 10:25:1 medicinal cannabis product, and a treatment phase of 28 days, with follow-up at 2 weeks after cessation of treatment. For the paracetamol/codeine arm, the dose of paracetamol/codeine will be used in accordance with the prescribing information for 28 days. Clinical response will be measured daily, and rescue medication (e.g., opioid analgesic) allowed as required.

Clinical response will be measured according to the primary efficacy assessment tool of the Numerical Rating Scale (NRS) of pain intensity, as described elsewhere herein. Secondary efficacy assessment tools will also be utilized, including measurement of physical functioning (e.g., Roland Morris Disability Questionnaire), sleep (e.g., Pittsburgh Sleep Quality Index), emotional function (e.g., Hospital Anxiety and Depression Scale or Depression Anxiety Stress Scale), quality of life (e.g., Euroqol 5D or Short Form Medical Outcomes 12), and patient-reported Global Impression of Change, each of which have been described elsewhere herein.

The co-primary end-points of the study are the change in pain intensity from baseline over previous week of treatment (at week 4), and the use of rescue medication (if any). The secondary end-points relate to the secondary efficacy assessment tools and include change in pain intensity from baseline over previous week of treatment for weeks 1, 2 and 3; responder rate for 30% reduction in mean pain intensity from baseline over previous week of treatment for week 4 as assessed by NRS; responder rate for 50% reduction in pain intensity from baseline over previous week of treatment for week 4 as assessed by NRS; change from baseline in sleep quality; change in baseline in physical functioning; change in baseline in emotional function; change in baseline in quality of life; and patient-reported global impression of change.

Blood samples will also be taken from a subset of patients (n=10 per group) at various doses of the 10:25:1 medicinal cannabis product at the start and end of the treatment phase for analysis of plasma/serum content of CBD, linalool, and/or THC to assess bioavailability and pharmacokinetic parameters.

The dose titration phase will be performed as set out in Table 13.

TABLE 1
Cannabidiol and related cannabinoids

| | | Chemical |
|---|---|---|
| | | properties/ |
| | | [M + H]⁺ ESI |
| Name | Structure | MS |
| cannabidiol (CBD) | 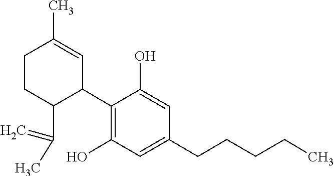 [SMILES: C=C(C)C1CCC(C)=CC1c1c(O)cc(CCCCC)cc1O] | decarboxylation product of CBDA m/z 315.2319 |
| cannabidiolic acid (CBDA) | 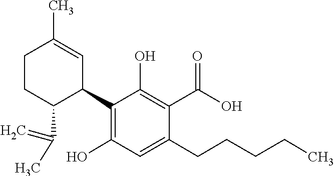 [SMILES: C=C(C)[C@@H]1CCC(C)=C[C@H]1c1c(O)cc(CCCCC)c(C(=O)O)c1O] | m/z 359.2217 |
| cannabigerolic acid (CBGA) | 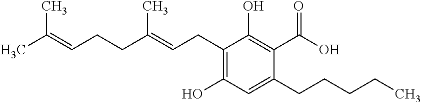 [SMILES: CCCCCc1cc(O)c(C/C=C(\C)CCC=C(C)C)c(O)c1C(=O)O] | m/z 361.2373 |

TABLE 2
Tetrahydrocannabinol and related cannabinoids

| | | Chemical |
|---|---|---|
| | | properties/ |
| | | [M + H] ⁺ ESI |
| Name | Structure | MS |
| Δ-9- tetrahydrocannabinol (THC) | 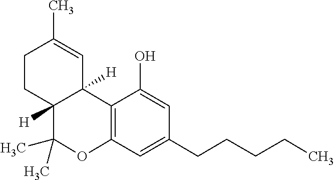 [SMILES: CCCCCc1cc(O)c2c(c1)OC(C)(C)[C@@H]1CCC(C)=C[C@@H]21] | Psychoactive, decarboxylation product of THCA m/z 314.46 |
| Δ9-tetrahydrocannabinolic acid (THCA) | 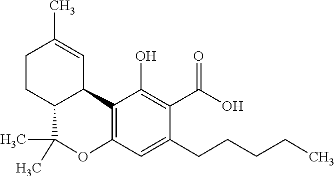 [SMILES: CCCCCc1cc2c(c(O)c1C(=O)O)[C@@H]1C=C(C)CC[C@H]1C(C)(C)O2] | m/z 359.2217 |
| cannabigerolic acid (CBGA) | 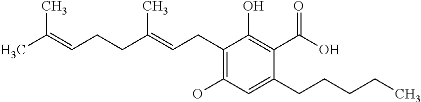 [SMILES: CCCCCc1cc(O)c(C/C=C(\C)CCC=C(C)C)c(O)c1C(=O)O] | m/z 361.2373 |

TABLE 3
Anti-inflammatory assays and positive controls

| | Plating | | Incubation | Kit | Positive |
|---|---|---|---|---|---|
| Assay | time (h) | Stimulation | time (h) | supplier | control |
| NO | 48 | LPS | 18 | Griess | 1400 W |
| | | (50 mg/mL) | | Reagent | (Cayman) |
| TNFα | 48 | LPS | 18 | Peprotech | Dexamethasone |
| | | (50 mg/mL) | | (USA) | (Sigma) |
| IL-6 | 48 | LPS | 18 | Peptrotech | Dexamethasone |
| | | (50 mg/mL) | | (USA) | (Sigma) |

TABLE 4
IC₅₀ for NO inhibition by CBD, THC and linalool

| | CBD | THC | Linalool | 1400 W |
|---|---|---|---|---|
| IC50 (μg/mL) | 1.4 | 6.5 | >240 | 1.2 |
| 95% confidence interval IC50 | 1.1-1.9 | 6.0-7.2 | Wide | 1.1-1.3 |
| (μg/mL) | | | | |

TABLE 5
NO assay IC₅₀ and CI results

| | Graph- | | | | | |
|---|---|---|---|---|---|---|
| | Pad | | Compusyn | | | |
| | IC₅₀ | | IC₅₀ | CI | CI | CI |
| | (μg/mL) | 95% CI | (μg/mL) | (IC₅₀) | (IC₇₅) | (IC₉₀) |
| CBD | 1.4 | 1.1-1.9 | 1.4 | | | |
| THC | 6.5 | 6.0-7.2 | 6.5 | | | |
| Linalool | >240 | — | >240 | | | |
| C:T (25:10) | 1.5 | 1.3-1.7 | 1.5 | 0.8 | 0.6 | 0.5 |
| C:T:L | 1.7 | 1.5-1.8 | 1.7 | 0.7 | 0.7 | 0.5 |
| (25:10:1) | | | | | | |
| C:T:L | 1.3 | 1.2-1.5 | 1.3 | 0.7 | 0.6 | 0.5 |
| (25:10:2) | | | | | | |
| C:T:L | 1.3 | 1.2-1.5 | 1.3 | 0.7 | 0.5 | 0.4 |
| (25:10:5) | | | | | | |
| C:T:L | 1.4 | 1.2-1.5 | 1.4 | 0.6 | 0.5 | 0.4 |
| (25:10:7.5) | | | | | | |
| C:T:L | 1.2 | 1.0-1.4 | 1.2 | 0.5 | 0.4 | 0.4 |
| (25:10:10) | | | | | | |
| C:T:L | 1.4 | 1.2-1.5 | 1.4 | 0.6 | 0.5 | 0.4 |
| (25:10:12.5) | | | | | | |
| C:T:L | 1.2 | 1.3-1.7 | 1.5 | 0.6 | 0.5 | 0.4 |
| (25:10:15) | | | | | | |

TABLE 6
Most active and synergistic NO assay combinations

| | | | | Increase in |
|---|---|---|---|---|
| | | | | potency of |
| | Compusyn | | Concentration | CBD in |
| | IC₅₀ | CI | of CBD at | combination |
| | (μg/mL) | (IC₅₀) | IC50 (μg/mL) | (%) |
| CBD | 1.4 | | | |
| THC | 6.5 | | | |
| Linalool | >240 | | | |
| C:T (25:10) | 1.5 | 0.8 | 1.1 | 31 |
| C:T:L (25:10:1) | 1.7 | 0.7 | 1.2 | 19 |
| C:T:L (25:10:2) | 1.3 | 0.7 | 0.9 | 59 |
| C:T:L (25:10:5) | 1.3 | 0.7 | 0.8 | 72 |
| C:T:L (25:10:7.5) | 1.4 | 0.6 | 0.8 | 70 |
| C:T:L (25:10:10) | 1.2 | 0.5 | 0.7 | 110 |
| C:T:L (25:10:12.5) | 1.4 | 0.6 | 0.7 | 90 |
| C:T:L (25:10:15) | 1.5 | 0.6 | 0.8 | 87 |

TABLE 7
IC₅₀ for TNF-α inhibition by CBD, THC and linalool

| | CBD | THC | Linalool | Dexamethasone |
|---|---|---|---|---|
| IC50 (μg/mL) | 4.6 | 44 | >240 | 2.7 |
| 95% confidence interval IC50 | 3-12 | Wide | Wide | 0.9-9.1 |
| (μg/mL) | | | | |

TABLE 8
TNF-α inhibition IC₅₀ and CI results

| | Graph- | | | | | |
|---|---|---|---|---|---|---|
| | Pad | | Compusyn | | | |
| | IC₅₀ | | IC₅₀ | CI | CI | CI |
| | (μg/mL) | 95% CI | (μg/mL) | (IC₅₀) | (IC₇₅) | (IC₉₀) |
| CBD | 4.6 | 3-12 | 4.6 | | | |
| THC | 44 | Wide | 45 | | | |
| Linalool | >240 | — | >240 | | | |
| C:T (25:10) | 6.7 | 3.6-40 | 6.8 | 1.1 | 1.8 | 3.0 |
| C:T:L | 5.7 | 3.6-22 | 5.7 | 0.9 | 0.8 | 0.8 |
| (25:10:1) | | | | | | |
| C:T:L | 5.8 | 3.7-31 | 5.8 | 0.9 | 0.7 | 0.5 |
| (25:10:2) | | | | | | |
| C:T:L | 5.5 | 3.0-26 | 5.1 | 0.7 | 1.9 | 4.8 |
| (25:10:5) | | | | | | |
| C:T:L | 5.5 | 3.8-13 | 5.6 | 0.7 | 0.6 | 0.5 |
| (25:10:7.5) | | | | | | |
| C:T:L | 6.6 | Wide | 6.6 | 0.8 | 0.5 | 0.3 |
| (25:10:10) | | | | | | |
| C:T:L | 4.7 | 3.2-12 | 4.7 | 0.6 | 0.5 | 0.4 |
| (25:10:12.5) | | | | | | |
| C:T:L | 3.6 | 1.0-7.7 | 5.2 | 0.6 | 0.3 | 0.1 |
| (25:10:15) | | | | | | |

TABLE 9
Most active and synergistic TNF-α assay combinations

| | | | | Increase in |
|---|---|---|---|---|
| | | | | potency of |
| | Compusyn | | Concentration | CBD in |
| | IC₅₀ | CI | of CBD at | combination |
| | (μg/mL) | (IC₅₀) | IC50 (μg/mL) | (%) |
| CBD | 4.6 | | | |
| THC | 45 | | | |
| Linalool | >240 | | | |
| C:T (25:10) | 6.8 | 1.1 | 4.9 | −5 |
| C:T:L (25:10:1) | 5.7 | 0.9 | 4.0 | 16 |
| C:T:L (25:10:2) | 5.8 | 0.9 | 3.9 | 17 |
| C:T:L (25:10:5) | 5.1 | 0.7 | 3.2 | 44 |
| C:T:L (25:10:7.5) | 5.6 | 0.7 | 3.3 | 40 |
| C:T:L (25:10:10) | 6.6 | 0.8 | 3.7 | 25 |
| C:T:L (25:10:12.5) | 4.7 | 0.6 | 2.5 | 86 |
| C:T:L (25:10:15) | 5.2 | 0.6 | 2.6 | 77 |

TABLE 10
IC₅₀ for IL-6 inhibition by CBD, THC and linalool

| | CBD | THC | Linalool | Dexamethasone |
|---|---|---|---|---|
| IC50 (μg/mL) | 1.4 | 4.2 | >240 | 0.016 |
| 95% confidence interval | 1.1-1.9 | 3.4-5.3 | | 0.0021-0.053 |
| IC50 (μg/mL) | | | | |

TABLE 11
IL-6 assay IC₅₀ and CI results

| | Graph- | | | | | |
|---|---|---|---|---|---|---|
| | Pad | | Compusyn | | | |
| | IC₅₀ | | IC₅₀ | CI | CI | CI |
| | (μg/mL) | 95% CI | (μg/mL) | (IC₅₀) | (IC₇₅) | (IC₉₀) |
| CBD | 1.4 | 1.1-1.9 | 1.4 | | | |
| THC | 4.2 | 3.4-5.3 | 4.2 | | | |
| Linalool | >240 | — | >240 | | | |
| C:T (25:10) | 1.6 | 1.2-2.3 | 1.6 | 0.9 | 1.3 | 1.7 |
| C:T:L | 2.2 | 1.7-3.1 | 2.1 | 1.2 | 1.0 | 0.8 |
| (25:10:1) | | | | | | |
| C:T:L | 2.1 | 1.5-3.2 | 2.2 | 1.1 | 1.1 | 1.1 |
| (25:10:2) | | | | | | |
| C:T:L | 1.7 | 1.3-2.4 | 1.7 | 0.8 | 0.8 | 0.8 |
| (25:10:5) | | | | | | |
| C:T:L | 2.4 | 1.8-4.1 | 2.4 | 1.1 | 1.3 | 1.4 |
| (25:10:7.5) | | | | | | |
| C:T:L | 1.6 | 1.2-2.2 | 1.6 | 0.7 | 0.8 | 0.8 |
| (25:10:10) | | | | | | |
| C:T:L | 1.8 | 1.5-2.3 | 1.8 | 0.8 | 0.6 | 0.4 |
| (25:10:12.5) | | | | | | |
| C:T:L | 2.4 | 1.9-3.5 | 2.5 | 1.0 | 0.6 | 0.4 |
| (25:10:15) | | | | | | |

TABLE 12
Most active and synergistic IL-6 assay combinations

| | | | | Increase in |
|---|---|---|---|---|
| | | | | potency of |
| | Compusyn | | Concentration | CBD in |
| | IC₅₀ | CI | of CBD at | combination |
| | (μg/mL) | (IC₅₀) | IC50 (μg/mL) | (%) |
| CBD | 1.4 | | | |
| THC | 4.2 | | | |
| Linalool | >240 | | | |
| C:T (25:10) | 1.6 | 0.9 | 1.1 | 23 |
| C:T:L (25:10:1) | 2.1 | 1.2 | 1.5 | −4 |
| C:T:L (25:10:2) | 2.2 | 1.1 | 1.5 | −6 |
| C:T:L (25:10:5) | 1.7 | 0.8 | 1.1 | 32 |
| C:T:L (25:10:7.5) | 2.4 | 1.1 | 1.4 | −1 |
| C:T:L (25:10:10) | 1.6 | 0.7 | 0.9 | 58 |
| C:T:L (25:10:12.5) | 1.8 | 0.8 | 0.9 | 48 |
| C:T:L (25:10:15) | 2.5 | 1.0 | 1.3 | 12 |

TABLE 13
Dose titration

| | | | Paracetamol/codeine Arm | | | |
|---|---|---|---|---|---|---|
| | 10:25:1 MC Arm | | | Paracetamol + | Placebo Arm | |
| | | Placebo | Placebo | codeine | Placebo | Placebo |
| | Cybis | (T) | (O) | (T) | (O) | (T) |
| Day | 0.25 mL oil | | 0.25 mL oil | | 0.25 mL oil | |
| 1-2 | once daily in | | once daily in | | once daily in | |
| | the evening | | the evening | | the evening | |
| Day | 0.5 mL oil | | 0.5 mL oil | | 0.5 mL oil | |
| 3-4 | once daily in | | once daily in | | once daily in | |
| | the evening | | the evening | | the evening | |
| Day | 1.0 mL oil | | 1.0 mL oil | | 1.0 mL oil | |
| 5-6 | as once daily | | as once daily | | as once | |
| | in the | | in the | | daily in the | |
| | evening | | evening | | evening | |
| Day | 1.5 mL oil | | 1.5 mL oil | | 1.5 mL oil | |
| 7-8 | as 0.5 mL in | | as 0.5 mL in | | as 0.5 mL in | |
| | the morning | | the morning | | the morning | |
| | and 1.0 mL | | and 1.0 mL | | and 1.0 mL | |
| | in the | | in the | | in the | |
| | evening | | evening | | evening | |
| Day | 2.0 mL oil | 2 tablets | 2.0 mL oil | 1-2 tablets | 2.0 mL oil | 1-2 tablets |
| 9 | as 0.5 mL in | 4-6 | as 0.5 mL in | 4-6 hourly | as 0.5 mL in | 4-6 hourly |
| | the morning. | hourly | the morning. | (max 8 | the morning. | (max 8 |
| | and 1.5 mL | (max 8 | and 1.5 mL | tablets/d) | and 1.5 mL | tablets/d) |
| | in the | tablets/d) | in the | | in the | |
| | evening | | evening | | evening | |

T—tablet; O—oil.

1. A pharmaceutical composition comprising a cannabinoid and a terpene, wherein the cannabinoid is enriched for cannabidiol (CBD) and Δ-9-tetrahydrocannabinol (THC), or pharmaceutically acceptable salts thereof, and wherein the terpene is enriched for linalool, or a pharmaceutically acceptable salt thereof.

2. The pharmaceutical composition of claim 1, further comprising one or more pharmaceutically acceptable carriers, diluents and excipients.

3. The pharmaceutical composition of claims 1 or 2, comprising from about 1 mg/mL to about 100 mg/mL CBD.

4. The pharmaceutical composition of any one of claims 1 to 3, comprising from about 10 mg/mL to about 75 mg/mL CBD.

5. The pharmaceutical composition of any one of claims 1 to 4, comprising from about 0.1 mg/mL to about 100 mg/mL linalool.

6. The pharmaceutical composition of any one of claims 1 to 5, comprising from about 1 mg/mL to about 100 mg/mL THC.

7. The pharmaceutical composition of claim 6, comprising from about 5 mg/mL to about 30 mg/mL THC.

8. The pharmaceutical composition of any one of claims 1 to 7, comprising about 10 mg/mL THC, about 25 mg/mL CBD and about 1 mg/mL linalool.

9. The pharmaceutical composition of any one of claims 1 to 8, wherein the CBD is synthetic CBD.

10. The pharmaceutical composition of claim 9, wherein the synthetic CBD consists of the (−) CBD enantiomer.

11. The pharmaceutical composition of any one of claims 1 to 10, wherein the THC is synthetic THC.

12. The pharmaceutical composition of any one of claims 1 to 11, wherein the pharmaceutical composition is formulated for oral administration.

13. The pharmaceutical composition of any one of claims 1 to 12 for use in the treatment or prevention of an inflammatory condition.

14. The pharmaceutical composition of claim 13, wherein the inflammatory condition is selected from the group consisting of sepsis, traumatic injury, ischemia, asthma, burns, irritable bowel syndrome, Alzheimer's disease, cancer, major depression, arthritis, multiple sclerosis and pain.

15. The pharmaceutical composition of claim 14, wherein the inflammatory condition is pain.

16. The pharmaceutical composition of claim 15, wherein the pain is chronic pain.

17. The pharmaceutical composition of claim 16, wherein the chronic pain is chronic back and/or neck pain.

18. A method for the treatment or prevention of an inflammatory condition, the method comprising administering to a subject in need thereof an effective amount of the pharmaceutical composition of any one of claims 1 to 12.

19. The method of claim 18, wherein the inflammatory condition is selected from the group consisting of sepsis, traumatic injury, ischemia, asthma, burns, irritable bowel syndrome, Alzheimer's disease, cancer, major depression, arthritis, multiple sclerosis and pain.

20. The method of claim 19, wherein the inflammatory condition is pain.

21. The method of claim 20, wherein the pain is chronic pain.

22. The method of claim 21, wherein the chronic pain is chronic back and/or neck pain.

23. The method of any one of claims 18 to 22, wherein the pharmaceutical composition is administered to the subject twice per day.

24. The method of any one of claims 18 to 23, comprising administering to the subject the pharmaceutical composition as escalating daily doses comprise a first daily dose and a second daily dose, wherein the effective amount of the pharmaceutical composition administered as the second daily dose is greater than the effective amount of the pharmaceutical composition administered as the first daily dose.

25. The method of claim 24, further comprising administering to the subject a third daily dose, wherein the effective amount of the pharmaceutical composition administered as the third daily dose is greater than the effective amount of the pharmaceutical composition administered as the second daily dose.

26. The method of claim 25, further comprising administering to the subject a fourth daily dose and/or subsequent daily dose, wherein the effective about of the pharmaceutical composition administered as the fourth daily dose is greater than the effective amount of the pharmaceutical composition administered as the third daily dose.

27. The method of any one of claims 24 to 26, wherein the effective amount of the first daily dose comprises from about 10 mg to about 20 mg CBD, from about 0.1 mg to about 1 mg linalool, and from about 1 mg to about 10 mg THC.

28. The method of claim 27, wherein the effective amount of the first daily dose comprises about 12.5 mg CBD, about 0.5 mg linalool, and about 5 mg THC.

29. The method of any one of claims 24 to 28, wherein the effective amount of the second daily dose comprising from about 20 mg to about 30 mg CBD, from about 0.5 mg to about 5 mg linalool, and from about 5 mg to about 15 mg THC.

30. The method of claim 29, wherein the effective amount of the second daily dose comprises about 25 mg CBD, about 1 mg linalool, and about 10 mg THC.

31. The method of any one of claims 25 to 23, wherein the effective amount of the third daily dose comprises from about 40 mg to about 60 mg CBD, from about 1 mg to about 5 mg linalool, and from about 15 mg to about 25 mg THC.

32. The method of claim 31, wherein the effective amount of the third daily dose comprises about 50 mg CBD, about 2 mg linalool, and about 20 mg THC.

33. The method of any one of claims 26 to 32, wherein the effective amount of the fourth daily dose comprises from about 65 mg to about 80 mg CBD, from about 1 mg to about 10 mg linalool, and from about 25 mg to about 35 mg THC.

34. The method of claim 33, wherein the effective amount of the fourth daily dose comprises about 75 mg CBD, about 5 mg linalool, and about 30 mg THC.

35. Use of a cannabinoid and a terpene in the manufacture of medicament for the treatment or prevention of an inflammatory condition, wherein the cannabinoid is enriched for cannabidiol (CBD) and Δ-9-tetrahydrocannabinol (THC), or pharmaceutically acceptable salts thereof, and wherein the terpene is enriched for linalool, or a pharmaceutically acceptable salt thereof.
